(19) United States
(12) Patent Application Publication
(10) Pub. No.: US 2021/0156864 A1
(43) Pub. Date: May 27, 2021

(54) Diagnostic and Therapeutic Biomarkers in Human Cancers and Methods of Use Thereof

(71) Applicant: The University of Toledo, Toledo, OH (US)

(72) Inventors: Mahasin A. Osman, Toledo, OH (US); William James Antonisamy, Toledo, OH (US)

(73) Assignee: The University of Toledo, Toledo, OH (US)

(21) Appl. No.: 16/953,581

(22) Filed: Nov. 20, 2020

Related U.S. Application Data
(60) Provisional application No. 62/939,027, filed on Nov. 22, 2019.

(51) Int. Cl. G01N 33/574; G01N 33/50

(52) U.S. Cl. CPC G01N 33/57484; G01N 33/57415; G01N 33/5091

(57) ABSTRACT

Methods of analyzing a sample, diagnosing a cancer, and a treating a patient are described. Various markers for cancers, including triple negative breast cancer, are disclosed.

### RELATED APPLICATIONS

This application claims priority to U.S. Provisional Application No. 62/939,027 filed under 35 U.S.C. § 111(b) on Nov. 22, 2019, the disclosure of which is incorporated herein by reference in its entirety.

### STATEMENT REGARDING FEDERALLY SPONSORED RESEARCH

This invention was made with no government support. The government has no rights in this invention.

### BACKGROUND

Despite advances in the treatment of lung cancer, the overall survival rates remain low, calling for biomarker discovery to combat the disease. Currently, targeted therapy focuses on tyrosine kinase inhibitors (TKI) against molecules such as the epidermal growth factor receptor (EGFR). Also PDL-1 based therapy has made great strides. However, these approaches are hindered by the invariable development of resistance. Similarly, triple negative breast cancer (TNBC) is an aggressive, heterogeneous, and highly invasive disease that lacks any of the receptors commonly found in breast cancer, such as the hormone and growth factor receptors. As such, TNBC lacks diagnostic markers and treatment targets. There is a need in the art for new and improved diagnostic markers and therapeutic targets for cancers such as lung cancer and TNBC. Furthermore, there is a need for diagnostics and therapeutics that do not rely on a single gene in order to avoid the development of resistance because genes (or their products proteins) work within pathways. This invention uses groups of markers operating in pathway(s)

### SUMMARY

In a first aspect, described herein is a method of analyzing a sample that includes:

extracting a tissue sample from a patient; and;

analyzing the tissue sample for expression levels of two or more markers selected from the group consisting of: Bcl2, caspase-3, centrin-2, acetyl α-tubulin, γ-tubulin, NRF1, β-catenin, pERK1, ERK1/2, pMMNk1, MNK1, pJNK, JNK1, pAkt1, Akt1, IQGAP1, pIQGAP1, BRCA1, and 2a-ADR.

In certain embodiments, the method includes analyzing the tissue sample for expression levels of at least two of: Bcl2, caspase-3, and IQGAP1.

In certain embodiments, the method includes analyzing the tissue sample for expression levels of each of: centrin-2, pERK1, ERK1/2, MNK1, pAkt1, Akt1, IQGAP1, and BRCA1.

In certain embodiments, the method includes analyzing the tissue sample for expression levels of each of: acetyl α-tubulin, γ-tubulin, NRF1, β-catenin, pMMNk1, pJNK, JNK1, pIQGAP1, and 2a-ADR.

In certain embodiments, the method includes analyzing the tissue sample for expression levels of three, four, five, six, seven, eight, nine, or ten more of the markers.

In certain embodiments, IQGAP1 is mislocalized in the tissue sample, or aberrantly phosphorylated in the tissue sample.

In another aspect, described herein is a method of analyzing a sample, the method generally includes:

extracting a tissue sample from a patient;

analyzing the tissue sample for a change in IQGAP1 expression compared to a control; and,

analyzing the tissue sample for an expression level (or activation such as by cleavage of caspase 3) of at least one marker selected from the group consisting of: Bcl2, caspase-3, centrin-2, acetyl α-tubulin, γ-tubulin, NRF1, β-catenin, pERK1, ERK1/2, pMMNk1, MNK1, pJNK, JNK1, pAkt1, Akt1, IQGAP1, pIQGAP1, BRCA1, and 2a-ADR.

In certain embodiments, the change in IQGAP1 comprises one or more of: aberrant phosphorylation of IQGAP1; IQGAP1 localized in centrosomes; and, IQGAP1 mis-localized and/or aggregated in the cytoplasm of tissue sample compared to normal tissues.

In certain embodiments, the sample is analyzed for the presence of triple negative breast cancer (TNBC), and the method further comprises:

distinguishing between distinct variants of TNBC, wherein said distinct variants include at least Caucasian (CA) TNBC, and African American (AA) TNBC.

In another aspect, described herein is a method of treating a patient. Such method generally includes:

extracting a tissue sample from a patient;

analyzing the tissue sample for a change in IQGAP1 expression compared to a control of normal tissue, where said change in IQGAP1 expression in the tissue sample compared to the control is indicative of the patient having, or being likely to have, a cancer; and;

treating the patient with a drug that modulates expression of IQGAP1.

In certain embodiments, the change in IQGAP1 expression compared to the control comprises one or more of:

i) determining phosphorylation level of IQGAP1 to determine whether IQGAP1 is aberrantly phosphorylated in the tissue sample compared to a control of normal tissue; wherein aberrant phosphorylation of IQGAP1 in the tissue sample compared to the control is indicative of the patient having, or being likely to have, a cancer; and;

ii) localization of IQGAP1 to determine whether IQGAP1 is localized in centrosomes or is mislocalized or aggregated in cytoplasm of the tissue sample; wherein mislocalization of IQGAP1 in the tissue sample is indicative of the patient having, or being likely to have, a cancer.

In certain embodiments, the method can further include: analyzing the tissue sample for an expression level of at least one marker selected from the group consisting of altered expression (up or down) and activation by cleavage: Bcl2, caspase-3, centrin-2, acetyl α-tubulin, γ-tubulin, NRF1, β-catenin, pERK1, ERK1/2, pMMNk1, MNK1, pJNK, JNK1, pAkt1, Akt1, IQGAP1, pIQGAP1, BRCA1, and 2a-ADR.

In certain embodiments, the method includes further analyzing the tissue sample for expression levels of at least two of: Bcl2, caspase-3, and IQGAP1.

In certain embodiments, the cancer is triple negative breast cancer in a patient. Such method generally includes:

a) detecting the expression pattern of a set of markers in a sample from the patient, wherein the set of markers comprises two or more markers selected from the group consisting of: Bcl2, caspase-3, centrin-2, acetyl α-tubulin, γ-tubulin, NRF1, β-catenin, pERK1, ERK1/2, pMMNk1, MNK1, pJNK, JNK1, pAkt1, Akt1, IQGAP1, pIQGAP1, BRCA1, and 2a-ADR;

b) diagnosing the patient as needing a cancer therapy regimen when two or more of said markers are abnormally expressed or activated; and,

c) treating the patient with an immunotherapeutic or small molecule inhibitors that targets the set of markers expressed in the patient.

In certain embodiments, the treating comprises administering to the patient an effective amount of IQGAP1-IR-WW peptide or a drug with same effect.

In certain embodiments, the drug comprises at least one of a kinase inhibitor; an antidepressant, paclitaxel, vinorelbine, docetaxel, and vinblastine.

In another aspect, described herein is a method of identifying a patient as eligible for IQGAP1-directed therapy and administering the IQGAP1 directed therapy to a patient thereby identified as eligible. When the patient has triple negative breast cancer, the method generally includes:

fixing a tissue sample obtained from the patient, wherein the sample comprises cells of the cancer or carcinoma, contacting the fixed tissue sample with an anti-IQGAP1 antibodies;

detecting binding of the agent to the fixed tissue sample to determine whether IQGAP1 is expressed in the sample; and,

identifying the patient as eligible for IQGAP1-directed therapy based on the expression of IQGAP1 as compared to a control.

In certain embodiments, the method further includes: administering the IQGAP1-directed therapy to the patient identified as eligible, wherein the IQGAP1 directed therapy is therapy with an anti-IQGAP1 therapeutic agent.

In certain embodiments, the tissue sample expresses IQGAP1, and IQGAP1 expression is determined as a percentage of tumor cells in the sample expressing detectable IQGAP1 (sometimes it is a numerical score from 1-3 determined by blinded observer—same as the DACO scoring method for EGFR: 1 is normal, 2 is medium and 3 is high).

In certain embodiments, the method further includes determining a treatment protocol for the patient, wherein detectable expression of IQGAP1 is an indication that the treatment protocol include treatment with the IQGAP1-directed therapy.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIG. 1: Genetic mutations do not explain the effect of IQGAP1 on cancer promotion. IQGAP1 is a modular protein with each domain involved in a distinct cellular function. Domains relevant for the underlined reasons were sequenced. CHD; calponin homology domain; IR-WW: IQGAP1-repeats (IR) and tryptophan (WW) repeats mediate protein-protein interaction; IQ: four isoleucine and glutamine rich motifs binds Myosin and MLC1; GRD: RasGTPase activating protein-related domain; RGCT: RasGAP-C terminus (RGCT) domain; the critical Ser-1443 that mediate IQGAP1 cycling and activity is indicated; NLS: nuclear localization signal that mediates IQGAP1 nuclear shuttling; aPI: binds phospho-lipid PIP3 and may mediate membrane localization. Sites for microtubule (MT)-binding proteins CLIP170 and APC, and the cell-cell adhesion proteins E-cadherin and β-catenin are shown in far C-terminus. No mutations were found in the cancer cell lines used in the examples herein, which is a deviation from a prior belief of a mutation in gastric cancer.

FIGS. 2A-2B: IQGAP1 is required for cancer cell proliferation.

FIG. 2A: Immuno-blot demonstrating reduction of IQGAP1's level by ˜90% in cancer cells transfected with IQGAP1's shRNAs vs. scramble control.

FIG. 2B: Proliferation rates of the TNBC MDA-MB-468 (African American) and MDA-MB-231 (Caucasian) cells that were treated with control “scramble” shRNA or with a mixture of IQGAP1-shRNAs. Error bars are means+/−s.d. for n=3. Knockdown of IQGAP1 inhibits cancer cell proliferation.

FIGS. 3A-3B: The phosphorylation level of IQGAP1 defines different cancer variants.

FIG. 3A: Immunoprecipitation (IP) performed from total proteins from control (cntl) MCF10A cells, MDAMB-468 (468) or MDA-MB-231 (231) with IQGAP1 monoclonal antibodies and blotted with PKC substrate pan Phospho-Serine antibodies. Upper panels: level of active phospho-IQGAP1 (pIQGAP1). Lower panels: IQGAP1 in the total lysate was blotted to demonstrate equal input.

FIG. 3B: Quantification of IQGAP1's phosphorylation level. Band intensities were quantified from three blots. Error bars are means+/−s.d for n=3, *p<0.0001. IQGAP1 phosphotyrosine was not detected.

FIGS. 4A-4D: IQGAP1 controls centrosome size and number and accordingly defines cancer variants.

FIG. 4A: Upper panels, show that in normal mammary MCF10A cells, endogenous IQGAP1 localizes to the centrosome dot juxtaposed to the nucleus with the centrosome marker pericentrin.

FIG. 4A: Lower panels, show that expression of the dominant negative (DN) mutant IQGAP1IR-WW in MCF10A cells enlarges the centrosome size with multiple nuclei hooked to the large centrosome (unipolar centrosome).

FIG. 4B: Expression of dominant active IQGAP1-F leads to multiple (supernumerary or amplified) centrosomes where IQGAP1 co-localizes with the centrosome marker centrin.

FIG. 4C: In cervical cancer (HeLa) cells having multiple centrosomes, expression of the DN IQGAP1 inhibits cell abscission (daughter cell separation) leading to a unipolar centrosome with a large size and consequent inhibition of cell proliferation. Thus, introduction of IQGAP1-IR-WW peptide into cervical cancer cells that exhibit the phenotype found in CA patient abolishes this phenotype and arrests the growth of cancer cells.

FIG. 4D: The TNBC MDA-MB468 mimics the IQGAP1IR-WW DN mutant phenotype, whereas MDA-MB231 mimics the dominant active. IQGAP1 resides with BRCA1 on the multiple centrosomes of MDA-MB231 cells. Staining for IQGAP1 identifies two molecularly distinct TNBC cells: one marked by centrosome aberrations with amplified centrosomes, and another without.

FIGS. 5A-5D: Differential expression of the centrosome protein markers centrin, acetylated α-tubulin, and γ-tubulin.

FIG. 5A: Results of testing expression levels of the centrosome resident proteins by immunoblotting in comparison to expression levels of IQGAP1 and the centrosome marker BRCA1. While the level of the stabilizing acetylated α-tubulin was significantly lower in MDA-MB-468 compared to MDA-MB-231, the converse is true for the levels of g-tubulin and centrin.

FIG. 5B: ANOVA quantification of the expression levels of the centrosome markers in TNBC cell lines.

FIG. 5C: The expression level of same centrosome proteins in a lung cancer panel showing high expression of acetylated α-tubulin marker in CA male 5816 cell lines.

FIG. 5D: ANOVA quantification of centrosome/microtubule markers in the lung cancer cell panel shows a significant difference in centrosome markers expression among the different cancer cell lines.

FIGS. 6A-6D: Differential expression and activation of the MAPK Erk1, Mnk1; Akt1, and the stress signal JNK (FIG. 6A). The expression levels of total MAPK, Erk1/2, and Mnk1, as well as the level of the stress signal JNK and the proliferation signal Akt1 were compared to their phosphorylated levels in TNBC (FIG. 6A cont.). The levels of total and phosphorylated kinases were quantified in reference to control actin and compared to control normal cells. As shown, both total and phosphorylated levels varied according to cell line.

FIG. 6B: Expression levels of total and phosphorylated MAPK, Akt1, and their downstream signaling molecules in a panel of six lung cancer cell lines.

FIGS. 6C-6D: Quantification of the levels in FIG. 6B shows significant differences in the expression and phosphorylation levels. This pattern presents these kinases as diagnostic markers and therapeutic targets in defined TNBC and lung cancer with application to other types of carcinomas.

FIGS. 7A-7D: Differential expression of the downstream transcription factors Nrf1 and β-catenin. Expression levels of the transcription factors Nrf1 and β-catenin were compared in TNBC with actin as a standard.

FIG. 7A: While β-catenin is highly expressed in the MDA-MB-468 and undetected in MDA-MB-231, Nrf1 is highly expressed in both cancer cell lines compared to control.

FIG. 7B: Differential expression level of the transcription factor Nrf1 in a panel of lung cancer cell lines as compared to IQGAP1. For example, both Nrf1 and β-catenin were highly expressed in 5816 and much less in 5810. IQGAP1 expression level also varies among cell lines.

FIGS. 7C-7D: Quantification of the protein levels demonstrating significant variations. Thus, Nrf1 and β-catenin levels can define specific cases (personalized) and serve as therapeutic targets.

FIGS. 8A-8D: Differential expression of upstream 2-α adrenergic receptor (2-AAR or 2α ADR). As a possible oncoprotein, the expression level of 2-AAR was tested by immunoblotting in the TNBC and lung cancer cell lines, comparing it to normal cells counterpart.

FIG. 8A: The receptor was highly expressed in the TNBC MDA-MB 468, but less so in MDA-MB 231 and control cells.

FIG. 8B: Quantification of the 2a ADR level in TNBC showing significant increase in MDA-MB 468 cells.

FIG. 8C: Expression level of 2a ADR is much less in the 5810 (AA male) lung cancer cell lines compared to control and to 5816 (CA male) cell lines.

FIG. 8D: Quantification of 2a ADR level in lung cancer cell panel shows significant differences among the members of the panel. This pattern provides a tool for diagnostics and personalized targeting via repurposing antidepressants as anticancer therapeutics.

FIGS. 9A-9D: IQGAP1 physically associates with BRCA1, Nrf1, Mnk1, and 2-AAR. Using immunoprecipitation (IP), physical interactions of IQGAP1 with the different markers in both TNBC and lung cancer were detected, both ways. Representative blots are shown.

FIG. 9A: Example for IQGAP1-BRCA1 interaction in TNBC cell lines.

FIG. 9B: Mnk1-IQGAP1 interaction in lung cancer cell lines.

FIGS. 9C-9D: IQGAP1 interaction with Nrf1 and ADR in both TNBC and lung cancer. Thus, IQGAP1 forms a complex with these biomarkers, demonstrating the existence of IQGAP1-specific pathways.

FIGS. 10A-10C: IQGAP1 and β-catenin are stabilized in the nuclei of cancer cells.

FIG. 10A: Immunoblot showing that unlike normal cells, IQGAP1 and β-catenin are found more in the nuclei of cancer cells. However, IQGAP1 level appears lower in these cancer cells.

FIGS. 10B-10C: Statistical quantification done after normalizing against actin control levels as well as against normal MCF10A mammary cells. Vinculin was blotted as a cytoplasmic marker to demonstrate efficient fractionation.

FIGS. 11A-11C: IQGAP1 colocalizes with BRCA1 and affects BRCA1 subcellular distribution.

FIG. 11A: Colocalization on multiple centrosomes.

FIG. 11B: In the upper two panels, IQGAP1-BRCA1 localizes to the nuclear envelope (arrow) where BRCA1 distributes approximately equally to the cytoplasm and nucleus.

FIG. 11C: In the upper panels, shows negative (inactive) mutants of IQGAP1 affects BRCA1 subcellular distribution and they co-localize in aggregates in the cytoplasm, arrow.

FIGS. 12A-12G: Comparison of localization and expression of IQGAP1 and BRCA1 [brown color] in human tissues.

FIG. 12A: Normal tissue.

FIG. 12B: Caucasian (CA) triple negative breast cancer (TNBC).

FIG. 12C: African American (AA) TNBC tissue.

FIGS. 12D-12F: Same tissues probed with BRCA1. Results: IQGAP1 is found in the plasma membrane (cell peripheries) in normal tissues, in CA tissues is perinuclear (in nuclear envelope), and in aggregates in the cytoplasm. In AA, IQGAP1 is dispersed in the cytoplasm. BRCA1 is found, albeit less expressed in normal tissues as expected, both in the nucleus and cytoplasm in normal tissues and found in cytoplasmic aggregates in cancer similar to IQGAP1.

FIG. 12G: These results are similar to the pattern in mutant cell culture where IQGAP1 and BRCA1 co-localize in cytoplasmic aggregates or on nuclear envelope in corresponding cells.

FIG. 13: Table 1, summarizing differential expression and/or phosphorylation levels of pathway markers in accordance with the present disclosure.

FIGS. 14A-14B: Results of testing expression levels of IQGAP1-signaling pathway in preclinical animal model by immunoblotting: IQGAP1, pAKT1, pGSK3α/β, pJNK1, Cytochrome-C, cleaved caspase-3. Bcl2 level expression levels were evaluated in control wild type (WT) mice and mice where iqgap1 gene was deleted from the chromosome, herein known as iqgap1 knockout (KO), after introduction of IQGAP-F or MDA-MB-231 TNBC cells in the mice. Immunoblots of protein expression for IQGAP1, pAKT1, pGSK3α/β, pJNK1, Cytochrome-C, and cleaved caspase-3, respectively, blotted against actin as loading control and comparing extracts from WT and KO. The expression levels of the markers varied significantly with the introduction of MDA-MB-231 TNBC cell lines into mice. This demonstrates that presence or absence of iQGAP1 has an effect and it modulates that markers thus providing evidence strong for suitability as diagnostic or therapeutic targets.

FIGS. 15A-15F: Statistical quantification of the two immunoblots in FIG. 14. The bands for each marker were quantified by densitometry against actin as control and significance calculated. Quantification provided significant differences in the expression levels of the tested markers depending on presence (WT) or absence (KO) of IQGAP1.

FIG. 16: Statistical quantification of the protein expression for Bcl2: This demonstrates that absence of iQGAP1 upon injection of MDA-MB-231 TNBC cell lines highly elevates the level of the oncoprotein Bl2. Overall these data demonstrates that IQGAP1 modulates the level of the tested markers to maintain cell homeostasis. Dysfunction of iQGAP1 leads to cancer development by deregulating these markers.

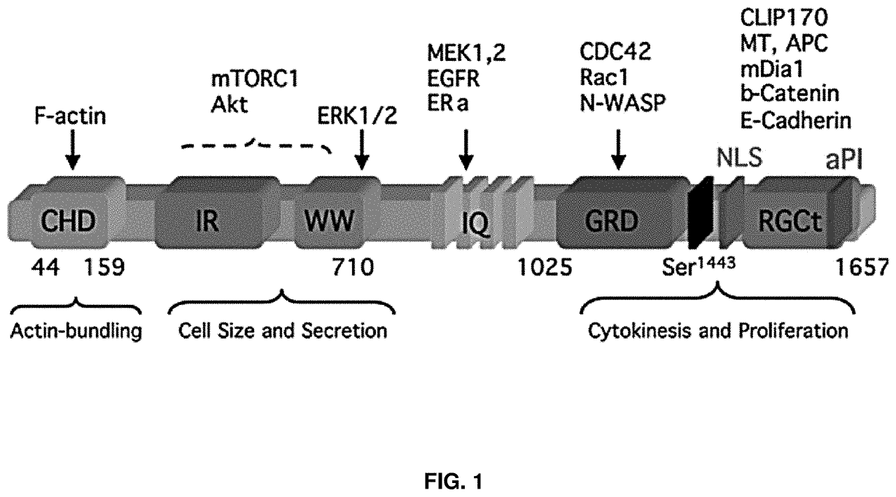

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

### Definitions

As used herein, including the claims, the singular forms “a,” “an,” and “the” include plural references, unless the content clearly dictates otherwise, and are used interchangeably with “at least one” and “one or more.”

The term “about” represents an insignificant modification or variation of the numerical values such that the basic function of the item to which the numerical value relates is unchanged.

The terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

The term “sample” as used herein includes any biological specimen obtained from a patient. Samples include, without limitation, whole blood, plasma, serum, red blood cells, white blood cells (e.g., peripheral blood mononuclear cells), ductal lavage fluid, nipple aspirate, lymph (e.g., disseminated tumor cells of the lymph node), bone marrow aspirate, saliva, urine, stool (i.e., feces), sputum, bronchial lavage fluid, tears, fine needle aspirate (e.g., harvested by random periareolar fine needle aspiration), any other bodily fluid, a tissue sample (e.g., tumor tissue) such as a biopsy of a tumor (e.g., needle biopsy) or a lymph node (e.g., sentinel lymph node biopsy), a tissue sample (e.g., tumor tissue) such as a surgical resection of a tumor, and cellular extracts thereof. In some embodiments, the sample is whole blood or a fractional component thereof such as plasma, serum, or a cell pellet. In certain instances, the sample is obtained by isolating circulating cells of a solid tumor from whole blood or a cellular fraction thereof using any technique known in the art. In other embodiments, the sample is a formalin fixed paraffin embedded (FFPE) tumor tissue sample, e.g., from a solid tumor of the breast.

A “set” of markers, probes or primers refers to a collection or group of markers, probes, primers, or the data derived therefrom, used for a common purpose (e.g., assessing an individual's risk of developing cancer). Frequently, data corresponding to the markers, probes or primers, or derived from their use, is stored in an electronic medium. While each of the members of a set possess utility with respect to the specified purpose, individual markers selected from the set as well as subsets including some, but not all of the markers, are also effective in achieving the specified purpose.

“Specimen” as used herein can refer to material collected for analysis, e.g., a swab of culture, a pinch of tissue, a biopsy extraction, a vial of a bodily fluid e.g., saliva, blood and/or urine, etc. that is taken for research, diagnostic or other purposes from any biological entity.

Specimen can also refer to amounts typically collected in biopsies, e.g., endoscopic biopsies (using brush and/or forceps), needle aspirate biopsies (including fine needle aspirate biopsies), as well as amounts provided in sorted cell populations (e.g., flow-sorted cell populations) and/or micro-dissected materials (e.g., laser captured micro-dissected tissues).

“Sample” as used herein can refer to specimen material used for a given assay, reaction, run, trial and/or experiment. For example, a sample may comprise an aliquot of the specimen material collected, up to and including all of the specimen. As used herein the terms assay, reaction, run, trial and/or experiment can be used interchangeably.

Certain methods may involve the use of a normalized sample or control that is based on one or more breast cancer samples that are not from the patient being tested. Methods may also involve obtaining a biological sample comprising breast cancer cells from the patient or obtaining a breast cancer sample.

Methods may further comprise assaying nucleic acids or testing protein expression in the breast cancer sample. In some embodiments, assaying nucleic acids comprises the use of polymerase chain reaction (PCR), microarray analysis, next generation RNA sequencing, any methods known in the art, or a combination thereof. In further embodiments, testing protein expression comprises ELISA, RIA, FACS, dot blot, Western Blot, immunohistochemistry, antibody-based radioimaging, mass spectroscopy, any methods known in the art, or a combination thereof.

In further embodiments, methods may comprise recording the expression level or the prognosis score in a tangible medium or reporting the expression level or the prognosis score to the patient, a health care payer, a physician, an insurance agent, or an electronic system.

The terms “overexpress”, “overexpression”, “overexpressed”, “up-regulate”, or “up-regulated” interchangeably refer to a biomarker that is transcribed or translated at a detectably greater level, usually in a cancer cell, in comparison to a non-cancer cell or cancer cell that is not associated with the worst or poorest prognosis. The term includes overexpression due to transcription, post transcriptional processing, translation, post-translational processing, cellular localization, and/or RNA and protein stability, as compared to a non-cancer cell or cancer cell that is not associated with the worst or poorest prognosis.

Overexpression can be detected using conventional techniques for detecting mRNA (i.e., RT-PCR, PCR, hybridization) or proteins (i.e., ELISA, immunohistochemical techniques, mass spectroscopy). Overexpression can be 10%, 20%, 30%, 40%, 50%, 60%, 70%, 80%, 90% or more (or any range derivable therein) in comparison to a normal cell or cancer cell that is not associated with the worst or poorest prognosis. In certain instances, overexpression is 1-fold, 2-fold, 3-fold, 4-fold 5, 6, 7, 8, 9, 10, or 15-fold or more higher levels of transcription or translation (or any range derivable therein) in comparison to a non-cancer cell or cancer cell that is not associated with the worst or poorest prognosis. The comparison may be a direct comparison where the expression level of a control is measured at the same time as the test sample or it may be a level of expression that is determined from a previously evaluated sample or an average of levels of expression of previously evaluated sample(s).

“Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. As used herein, “prognostic for cancer” means providing a forecast or prediction of the probable course or outcome of the cancer. In some embodiments, “prognostic for cancer” comprises providing the forecast or prediction of (prognostic for) any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer.

“Subject” or “patient” refers to any single subject for which therapy is desired, including humans, cattle, dogs, guinea pigs, rabbits, chickens, and so on. Also intended to be included as a subject are any subjects involved in clinical research trials not showing any clinical sign of disease, or subjects involved in epidemiological studies, or subjects used as controls. The terms “subject” and “patient” may be used interchangeably.

Several acronyms or abbreviations may be used herein for ease of description.

TNBC stands for triple negative breast cancer. The term “triple-negative” in the context used herein includes a tumor cell (e.g., a circulating tumor cell), a tumor, or a cancer such as triple-negative metastatic breast cancer (TNMBC) in which there is no detectable expression of estrogen receptor (ER), progesterone receptor (PR), or human epidermal growth factor receptor 2 (HER2).

Bcl2 (B-cell lymphoma 2) is an oncogene that normally acts as anti-apoptotic by controlling mitochondrial permeability and release of cytochrome C.

Caspase-3 protein is a member of the cysteine-aspartic acid protease (caspase) family that execute cell death; however in context of cancer it can transmit oncogenic C.

Centrin 2, also known as ascaltractin, is found in the centrosomes of many organisms and is a member of the highly conserved superfamily of calcium-binding EF-hand proteins.

2-alpha adrenergic receptor, and is referred to herein as 2A-ADR or 2AAR, is currently targeted as an anti-depressant.

Mnk1 is the mitogen-activated protein kinase (MAPK)-interacting serine/threonine kinase, acting downstream of ERK1/2.

ERK 1/2 is extracellular signal-regulated kinase 1/2, a member of MAPK.

Nrf1 is the nuclear respiratory factor 1, which is a member of the vertebrate Cap'n'Collar (CNC) leucine zipper transcription factors and is implicated in regulating antioxidant enzymes by binding to their antioxidant response element (ARE).

BRCA1 is breast cancer type 1, which is a tumor suppressor.

IQGAP1 is the IQ-containing guanine nucleotide activating protein 1, which is an effector of the Ras subfamily Cdc42 and Rac1, as well as possibly Ras and Rho.

General Description

IQGAP1 is a regulatory scaffold and a known oncoprotein that normally plays essential structural and signaling roles in the cell. It organizes the actin and microtubule cytoskeleton to regulate cell movement, cell division or adhesion, and it transduces signals emanating from surface receptors to the nucleus to control gene expression. IQGAP1 protein is regulated by addition or removal of phosphate groups (i.e. phosphorylation/dephosphorylation) on a serine residue in its C-terminus according to specific signals. This cycling changes the form of the protein to enable exclusive interactions with different proteins. Locking IQGAP1 in one form or the other is detrimental to the cell and causes different types of diseases, including cancer. Indeed, IQGAP1 overexpression associates with many carcinomas and has been proposed as a clinical target. However, its mechanism and unique pathway in each cancer must be defined for effective targeting, which the present disclosure provides. In contrast to the previously reported overexpression of IQGAP in cancers, in accordance with the present disclosure, it has been found that IQGAP1 is either underexpressed, aberrantly phosphorylated, or mislocalized in certain cancers, thus providing clinical tools as a diagnostic and therapeutic biomarker, and may allow for the repurposing of growth factors or kinase inhibitors to serve as treatments for cancers.

IQGAP1 regulates the activity of the mitogen protein kinase (MAPK) Erk1/2. Mnk1, also known as MKNK1-Mitogen-Activated Protein Kinase (MAPK)-interacting serine/threonine kinase—is a downstream target of Erk and has been implicated in regulating mRNA translation, oncogenesis, drug resistance, and inflammation, mostly through its major downstream effector, the cap binding eukaryotic initiation factor 4E (eIF4E). However, Mnk1's role in cancer development is incompletely defined. In accordance with the present disclosure, Mnk1 is a valuable marker/target that is overexpressed or hyper-phosphorylated in certain cancers but not others.

In animal cells, the centrosome is the microtubule (MT) organizing center (MTOC) that generates cytoskeleton MT, aster MT, as well as spindle MT that segregate the chromosomes equally to the two daughter cells. Normally, the centrosome divides only once per cell cycle to deliver the proper number of chromosomes to each daughter cell. Centrosome amplification (CA) widely associates with human malignancies and is a hallmark of cancer. CA is observed in 20-30% of cells overexpressing oncogenes or lacking tumor suppressors such as BRCA1. It is believed that CA represents an earlier step in tumorigenesis and contributes to tumor progression, however the molecular players that regulate centrosome numbers remain poorly defined. In accordance with the present disclosure, IQGAP1 resides on centrosomes, binds centrosome proteins, and regulates centrosome size and number. In its active form, IQGAP1 generates multiple centrosomes (increased number), whereas in its inactive form, IQGAP1 arrests centrosome division, holding it in a large size. Each of these phenotypes defines a distinct variant of TNBC or lung cancer. The present disclosure provides a method for early detection of cells with aberrant centrosomes (size or number) that may develop into cancer and progress into deadly metastasis.

In dividing somatic cells, CA often leads to transient formation of multipolar spindles. In normal cells, this event activates cell death programs to prevent chromosome missegregation that leads to aneuploidy and cancer or developmental defects. Cancer cells overcome this checkpoint, allowing the extra centrosomes to cluster together to form a spindle with just two poles, resulting in a bipolar division and an advantage to proliferate. Centrosome clustering inhibitors are available, some of which, such as the antifungal griseofulvin, are FDA-approved. In accordance with the present disclosure, existing drugs, such as kinase inhibitors, can be leveraged to target a specific subset of cancer defined by CA and/or altered IQGAP1 activity.

An important centrosome marker is the resident protein centrin. Centrin plays fundamental roles in centrosome structure and function, centriole duplication, regulation of cytokinesis, and global genome nucleotide excision repair. Many of these functions mirror those of IQGAP1 and support the finding herein that IQGAP1 and centrin physically interact and localize at the centrosome. In accordance with the present disclosure, altered interaction marks certain cancers but not others, thus allowing for targeted therapy.

Beside their role in cytoskeleton organization, microtubules serve as a signal transduction platform during cell division and have been targeted in cancer therapy. Acetylation of α-tubulin on lysine 40 (K40) [Acety-α-tubulinK40], is a well-known marker of stabilized microtubules, and has been implicated in the metastatic potential of breast cancer. On the other hand, increased expression or delocalization of g-tubulin from the centrosome to the cytoplasm is observed in breast cancer cell lines. In accordance with the present disclosure, acetyl-α-tubulin can be utilized as a marker and a personalized therapeutic target of specific cancers.

β-catenin is a component of centrosomes and of cell-cell adhesion where it forms a complex with IQGAP1, and it associates with human cancers. Mutant analyses of β-catenin indicate that it plays a role in centrosome amplification, but its mechanism of action is unknown, thus hindering utility as an anticancer target. IQGAP1 binds β-catenin to regulate cell-cell contacts (adheren cell junctions) and transcription. In accordance with the present disclosure, specific variants of cancer marked by aberrant β-catenin expression or localization are identified and can benefit from available or new targeted therapy, thus providing diagnostic and therapeutic tools.

In the examples herein, a plurality of markers that together provide a “molecular signature” enabling new classification of cancers that were previously grouped under a single subtype and treated with shotgun approaches is provided. Importantly, these markers can be utilized as common diagnostic tools for different types of carcinomas independent of organ site and useful for attenuating racial health disparity with regard to diagnosis and treatment.

The present disclosure provides methods of detecting neoplastic cells in tissues, molecular classification of neoplasm, and, advantageously, provides targets for treatment irrespective of organ site. Methods that involve measuring expression, activity, and subcellular location of signature markers are utilized in which increased or decreased expression or altered localization of the gene product compared to a standard or known level of expression associated with normal tissue is indicative of the presence of neoplastic cells of a specific type. These markers also constitute targets for therapy with existing FDA-approved drugs and inform devising more effective drugs. Importantly, the present disclosure provides classification (segmentation) of cancers thus allowing for personalized treatment with targeted therapy.

In certain embodiments examples described herein, genetic mutation was measured by Sanger sequencing of important regions of the gene known to promote cell proliferation, localization of the protein, or to mediate protein-protein interaction with important partners, leading to important module formation (scaffolding). The effect of a protein on centrosome amplification was measured by mutant analyses and localization studies with fluorescent probes and examined with super resolution microscope to detect locations in the cell in mutants and cancer cells as compared to normal cell counterparts. Involvement in promoting or sustaining cancer was evaluated by downregulating that protein via RNA-interference (RNAi).

Another embodiment of the present disclosure entails measuring gene expression of a panel of markers by isolating proteins from cancer cells and quantifying the amount of the markers, using specific antibodies for each of the markers. Correlation of the level of expression of a marker with its cellular activity can be measured by two methods, first by determining correct localization by isolating proteins from subcellular organelles using a biochemical fractionation method followed by detecting the presence of the markers with specific antibodies against known standards, and second by determining protein activities by measuring the level of phosphorylation with specific antibodies that differentiate between phosphorylated and unphosphorylated forms. In another embodiment, localization of the marker proteins in the subcellular compartments is visualized in cancer cells compared with normal cells as a standard by hybridizing/binding specific antibody against a protein marker followed by binding specific fluorescent probe to the antibody to visualize its location within the cells. Deviation from normal cells in terms of protein expression level, subcellular location, or activity as measured by phosphorylation of these proteins compared to a standard or known level is indicative of neoplastic cells of a specific class. By comparing cancer cells isolated from Caucasian and African American, the examples herein also provide methods for detecting racial health differences in cancer inception and progression, and offer therapeutic avenues to attenuating racial health disparities.

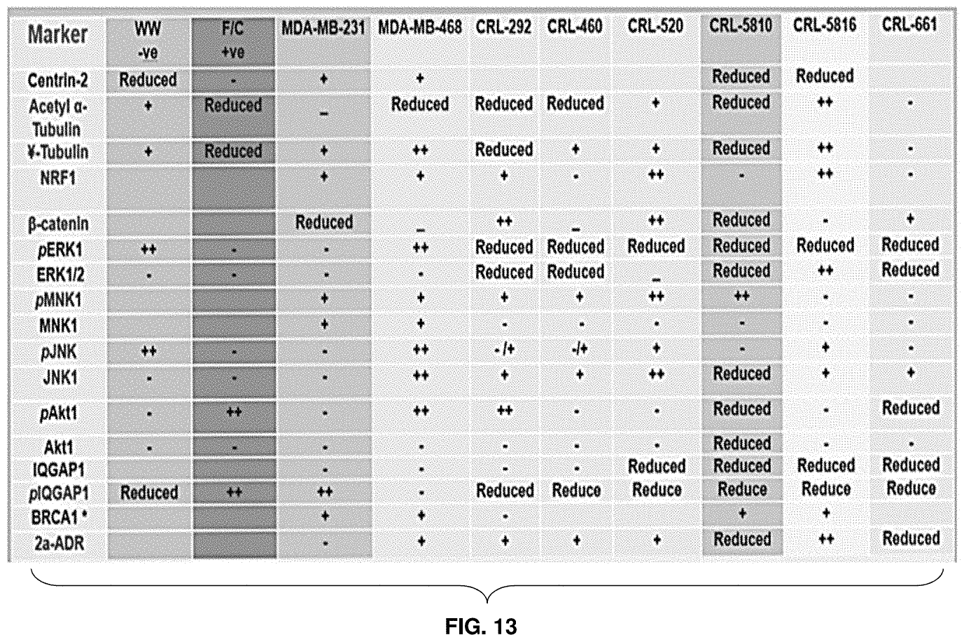

Table 1 (FIG. 13) shows various markers in accordance with the present disclosure. While Table 1 lists 18 markers, subsets of markers may be utilized. As one non-limiting example, Table 1 shows 7-9 markers in red that can be used for diagnosis and therapy. As another non-limiting example, the expression levels of tubulin, centrin, catenin, Mnk1, and Nrf1 are used together as markers for human cancer. A diagnostic kit based on biopsy staining [immunohistochemistry (IHC)] or immunoblot utilizing these markers is provided herein. Provided herein is a diagnostic kit comprising two or more biomarkers in cancer that can be used as treatment targets according to their status in the individual patient. These biomarkers have been tested in aggressive forms of cancer, namely, triple negative breast cancer (TNBC) and lung cancer, that lack diagnosis and treatment. A kit in accordance with the present disclosure may be based on biopsy staining (IHC) or immunoblotting. In some embodiments, the diagnostic kit may include 7-9 antibodies. However, other numbers of antibodies are possible and encompassed within the scope of the present disclosure. Microscopy and immunohistochemistry can identify localization of the markers.

The dysregulation of any particular marker shown in Table 1 indicates a different appropriate treatment. For example, if tubulin is over-expressed in the tissues of a patient, then a drug that targets tubulin may be administered to the patient as an appropriate treatment step to treat or prevent a cancer. Tubulin may be targeted, for instance, with the drug paclitaxel (Taxol®). As another example, if BRCA1 is aberrant, then an appropriate treatment step may be to administer a drug that targets PPAR as a way to target BRCA1 consequent aberrations. Mis-localization or quantified altered subcellular distribution of IQGAP1-BRCA1 defines certain TNBC.

The markers shown in Table 1 (FIG. 13) may also be used as singular markers for diagnosing cancers. For example, as seen in FIG. 13, the IQGAP1-interacting receptor α-ADR is differentially expressed in cancers.

Suitable treatments may be administered as part of any method described herein. In some embodiments, the treatment comprises administering IQGAP1-IR-WW peptide. Treatments other than the IR-WW-peptide may be administered. Specific inhibitors against phospho-IQGAP1, and antagonists or agonists for ADR, including antidepressants. Currently available antimicrotubules include paclitaxel (Taxol®), vinorelbine (Navelbine®), docetaxel (Taxotere®), and vinblastine (Velban®).

For a marker that is mis-localized or aberrantly phosphorylated, an appropriate treatment step may be to administer a kinase inhibitor. Kinase inhibitors include, but are not limited to, the drugs bosutinib, crizotinib, dasatinib, erlotinib, gefitinib, lapatinib, pazopanib, ruxolitinib, sunitinib, and vemurafenib.

The present disclosure provides markers for organ-inspecific epithelial cancers, and presents biomarkers in the EGFR pathway that can be developed into a kit that serves the dual role of diagnosis and personalized treatment. IQGAP1 scaffold being differentially under-expressed, phosphorylated, and/or mislocalized affects centrosome number and microtubule (MT) stability, and is therefore a marker for cancer. IQGAP1-interacting stress signal kinases are differentially expressed and/or phosphorylated in individual cancers of the same class or subtype. Downstream interacting transcription factors with a dual role in centrosome function are differentially expressed and localized. The present disclosure illustrates the aberrant expression of IQGAP1 in a specific pathway that controls microtubule dynamics and centrosome aberrations (i.e., cell division and proliferation), which is at the heart of cancer development and maintenance. Targeting pathways is an effective way to address the shortcomings of existing mono-therapies.

### EXAMPLES

Certain embodiments of the present invention are defined in the Examples herein. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

A multifaceted investigation to substantiate findings and provide effective methods for detecting different molecular classes of neoplasm via diagnostic markers that can double up as therapeutic targets was performed. Female TNBC and male lung cancer cell lines isolated from Caucasian and African American patients obtained from ATCC were used.

### Example 1—Mutation Analyses

While the MDA-MB 231, isolated from a Caucasian patient, and the MDA-MB-468, isolated from an African American patient, exhibit distinct morphological features, they are both classified as TNBC, a highly heterogeneous breast cancer subtype. IQGAP1 mutation/SNPs is occasionally seen in certain human cancers. To determine likely molecular differences in the two cell lines, genomic DNA sequencing was performed to examine possible presence of IQGAP1 mutations in the TNBC MDA-MB 231 and MDA-MB 468 cell lines that may explain the phenotypes observed in each cell type. Exons 18 constituting the WW domain, exon 36-38 constituting the aPI, and exons 33-36 constituting the NLS were selected. Genomic DNA was purified from cancer cells and normal cell counterparts. Targeted genes were amplified by PCR, using standard reaction conditions. Sanger sequencing of the gene indicated no mutation was found in any of the exons in the examined cancer cell lines, indicating that mutation of IQGAP1 gene does not account for the mechanism of IQGAP1 in these tumor cells.

### Example 2—RNAi-Mediated Knockdown

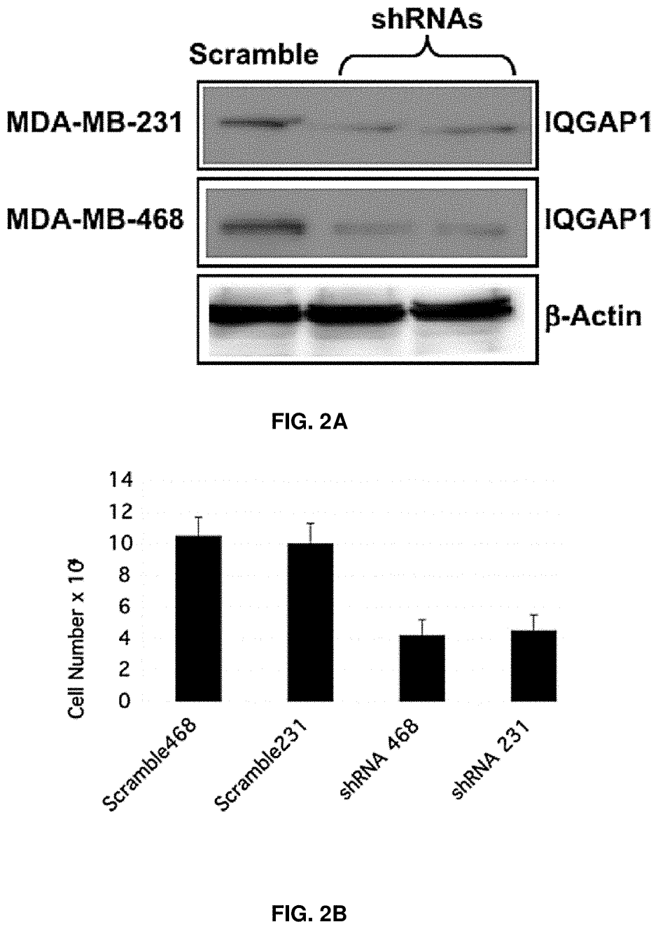

The requirement for IQGAP1 in the proliferation capacity of the TNBC cells was evaluated by RNA interference, which knocked down (decreased) IQGAP1 protein level by about 90%. A set of three antisense small hairpin RNAs (shRNAs) against human IQGAP1 were used along with control shRNAs that do not target IQGAP1 or any other gene, from Santa Cruz Biotech, following the manufacturer's protocol. After 48 h, the cells were counted for measuring proliferation or lysed for evaluating protein depletion by immunoblotting/Western Blot (see below for method). The results, shown in FIGS. 2A-2B, indicate that IQGAP1 is required for cell proliferation of cancer cells.

### Example 3—Phosphorylation (Activity) Level

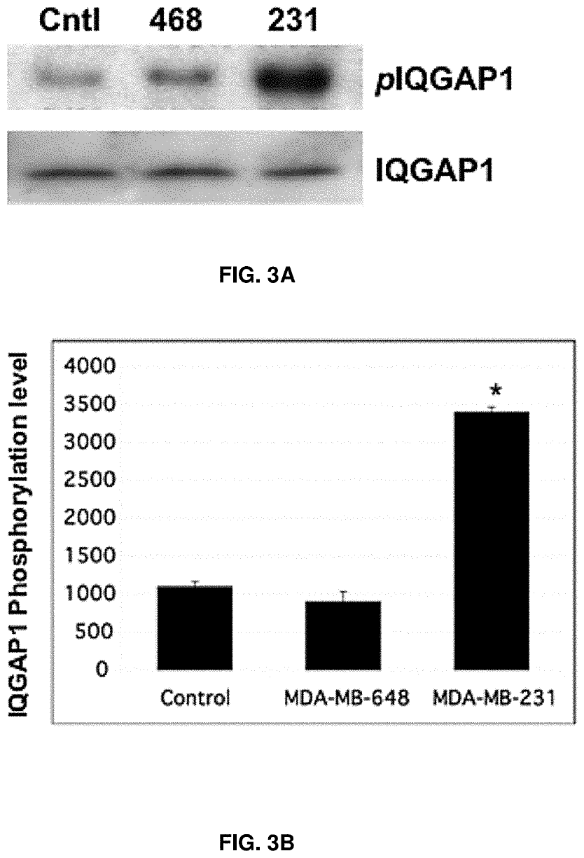

The expression level of IQGAP1 was measured in the TNBC cells vs. MCF10A, which represents normal mammary epithelia, and found that IQGAP1 protein level is not significantly changed. IQGAP1 phosphorylation on Serine 1443 at the C-terminus increases cell proliferation, causing cell transformation of normal cells. Therefore, the Serine phosphorylation in cancer vs. normal cells was measured. IQGAP1 was immunoprecipitated from total proteins isolated from cancer and control cells and blotted that with specific phosphoserine and phosphotyrosine antibodies obtained from Cell Signaling Biotechnology. While phosphotyrosine of IQGAP1 was not detected in any cell line, high level phosphoserine was detected only in MDA-MB 231 cells and not in the MDA-MB 468 cells, indicating the existence of two different mechanisms of IQGAP1-associated cancer in MDA-MB 231 vs. MDA-MB 468 cells. These results are shown in FIGS. 3A-3B.

### Example 4—Regulation of Centrosome Size and Number (Division)

Next, the mechanism of IQGAP1 was analyzed in the different cell lines and compared with IQGAP1 activating and inactivating mutants. Previously, it was established that different mutants of IQGAP1 created in the lab affect cell proliferation and migration, and therefore concluded that IQGAP1 regulates cells size and division. Here, how these mutants localize inside the cells was examined, using two methods: biochemical fractionation and super resolution confocal miscropy (SRM).

To conduct biochemical fractionation, nuclear and cytosolic fractionation was performed essentially as described previously. Briefly, ˜80% confluent cells were washed with cold PBS, then lysed on ice in a lysis buffer (20 mM HEPES, pH 7.2, 10 mM KCl, 2 mM MgCl₂, 0.5% Nonidet P40, 1 mM Na₃VO₄, 1 mM phenylmethylsulfonyl fluoride, 0.15 units ml-aprotinin or a protease inhibitor cocktail from Fisher) and homogenized by a tightly fitting Dounce homogenizer. The lysate was centrifuged at 1,500×g for 5 min to sediment the nuclei. The supernatant was then centrifuged at 15,000×g for 10 min at 4° C., and the resulting supernatant was saved as cytosolic fraction. The nuclear pellet was washed three times with lysis buffer and resuspended in the same buffer supplemented with 0.5 M NaCl to extract the nuclear proteins. The extracts were centrifuged at 15,000×g for 10 min, and the resulting supernatant was saved as nuclear fraction. Equal amounts of proteins (with GAPDH as loading control and Vinculin and PARP as fraction controls) were evaluated by Western blotting (immunoblotting) with IQGAP1 antibodies as described below, quantified by densitometry, using a BioRad GelDoc Imager and expressed as histograms, using Microsoft Excel Software. These experiments showed that while IQGAP1 is normally found both in the nucleus and cytoplasm, and introducing mutant IQGAP1 into cells displaces much of IQGAP1 into the cytoplasm. Therefore, the cytoplasmic location of IQGAP was examined using super resolution microscopy.

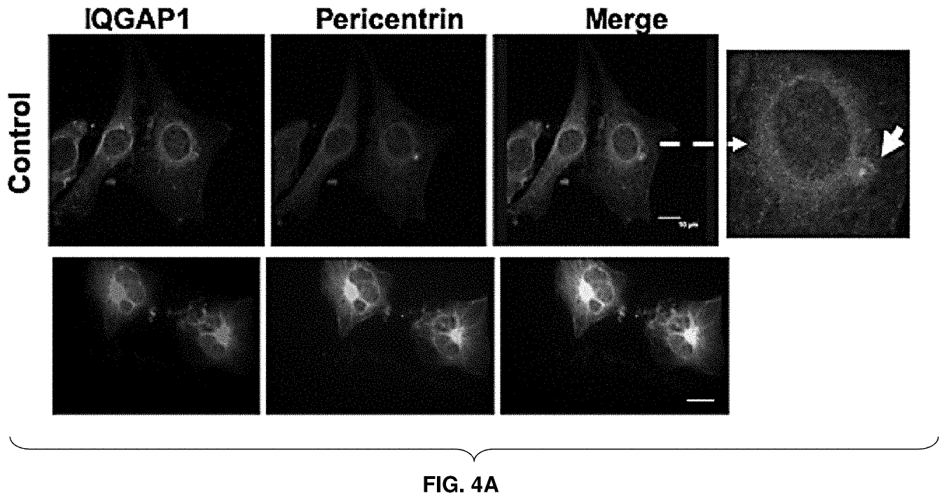

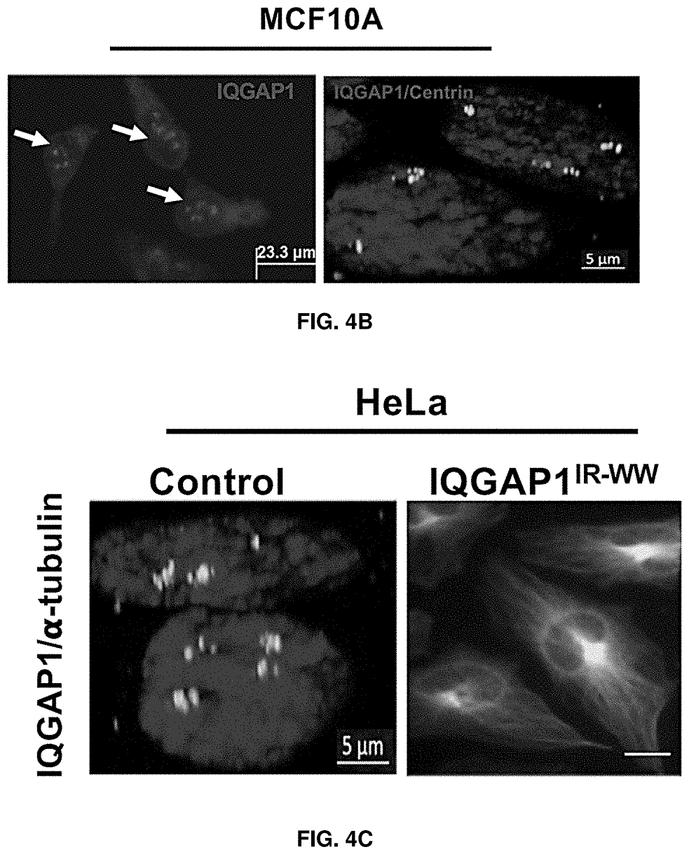

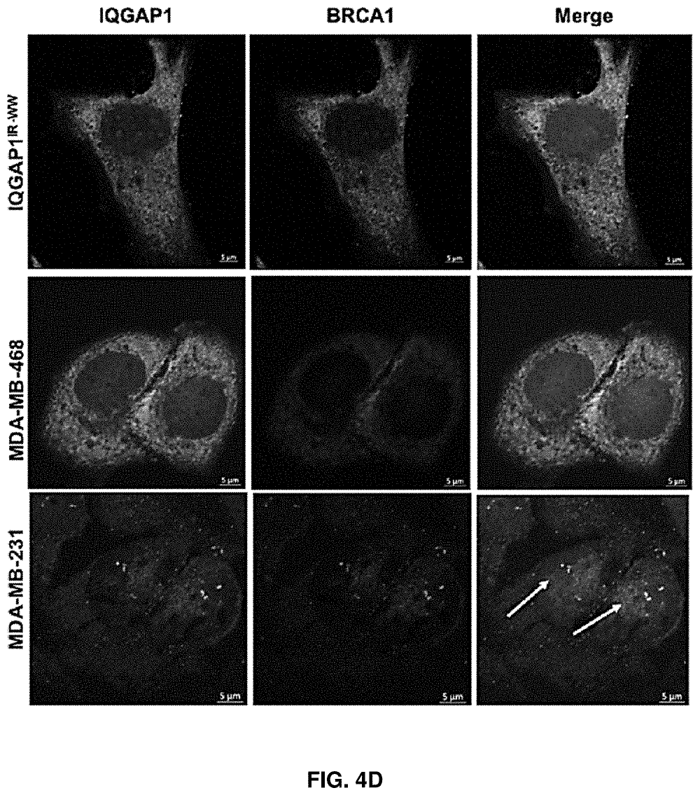

IQGAP1 localization in these mutants in comparison with normal and mutant and cancer cells was further visualized using super resolution microscopy, which reveals molecular level details inside the cells. Cells were cultured in multiple-chamber slides (Nalge, Nunc), washed with PBS and fixed in ˜20° C. methanol for 10 min, permeabilized in PBS containing 1% triton 100×, and blocked with 1 mg/ml BSA in PBS, incubated with primary or control antibodies followed by secondary (Texas Red, Alexa Fluor 555, or Alexa Fluor 488, Molecular Probe) for 1 hr. each at room temperature, and the nuclei were stained with DAPI (Sigma or Invitrogen). The centrosome was visualized with centrin, pericentrin, γ-tubulin antibodies, FITC-α-ubulin antibody (Sigma), or monoclonal-α-tubulin along with BRCA1, all obtained from Cell Signaling and/or Santa Cruz Biotech. The cells were imaged with a Zeiss LSM800 Laser Scanning Confocal Microscope with Airyscan and the images were composited in Adobe Photoshop and quantified as described below. This method revealed that while activating mutants of IQGAP1 caused multiple centrosomes on which IQGAP1 resided, the negative mutants (unphosphorylated) produced a single large centrosome with multiple nuclei attached to it (unipolar centrosome), indicating that IQGAP1 regulates centrosome number and size. The later phenotype also indicates dysfunction of cell abscission, which is the latest step of cytokinesis. When compared in TNBC cells, this method revealed that IQGAP1 localizes to multiple centrosomes in the MDA-MB-231 with BRAC1 similar to active IQGAP1, but CA was absent in the MDA-MB 468 cells similar to the negative mutants of IQGAP1. This method revealed two distinct mechanisms for IQGAP1 in cancer where one is associated with active IQGAP1 and CA and the other is associated with inactive IQGAP1 and a lack of centrosome amplification. It also shows that these TNBC cell lines can be classified into two distinct groups, according to the patient's race or to the centrosome aberration. These results are shown in FIGS. 4A-4C.

### Example 5—Differential Expression of Centrosome Marker Proteins as Biomarker/Target

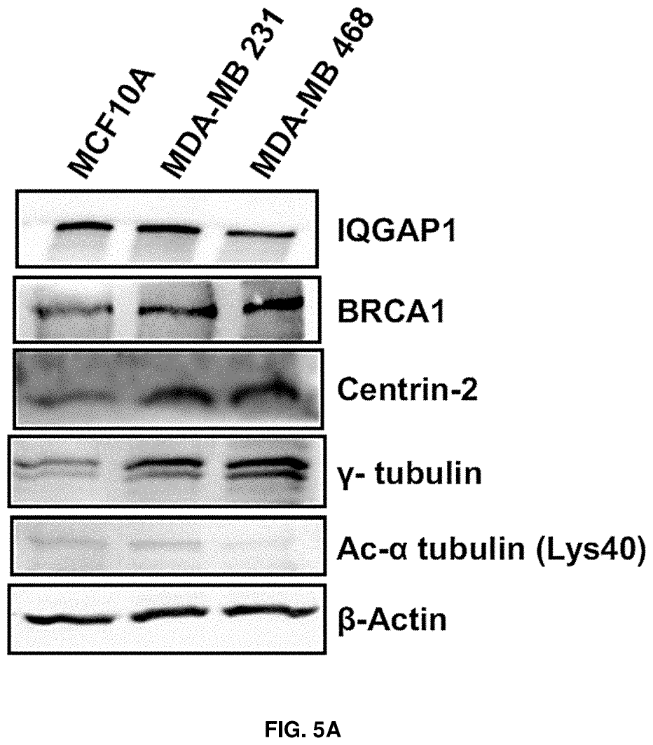

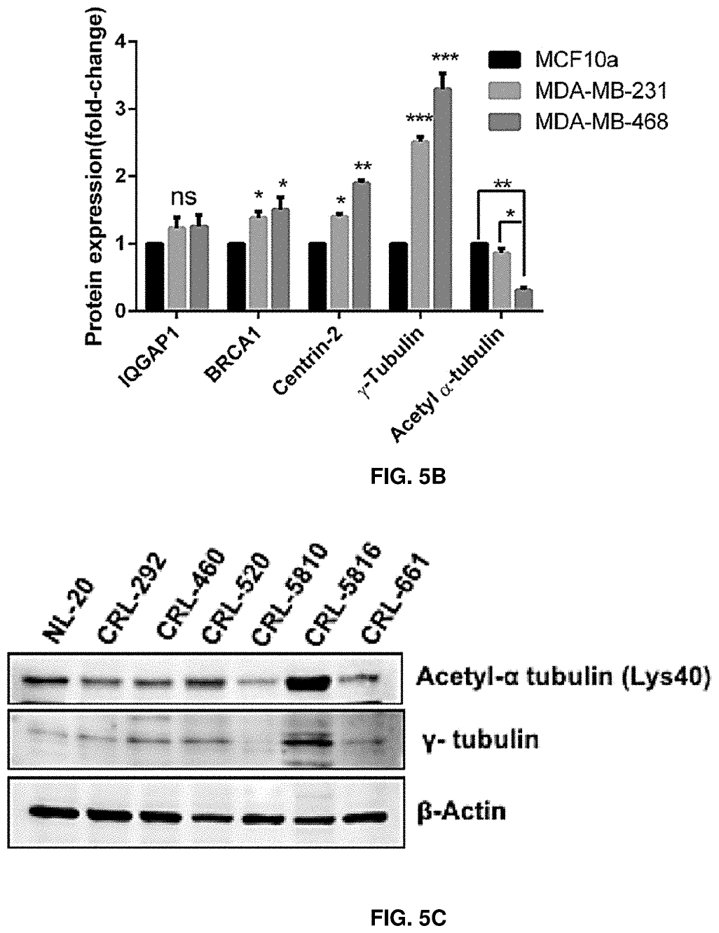

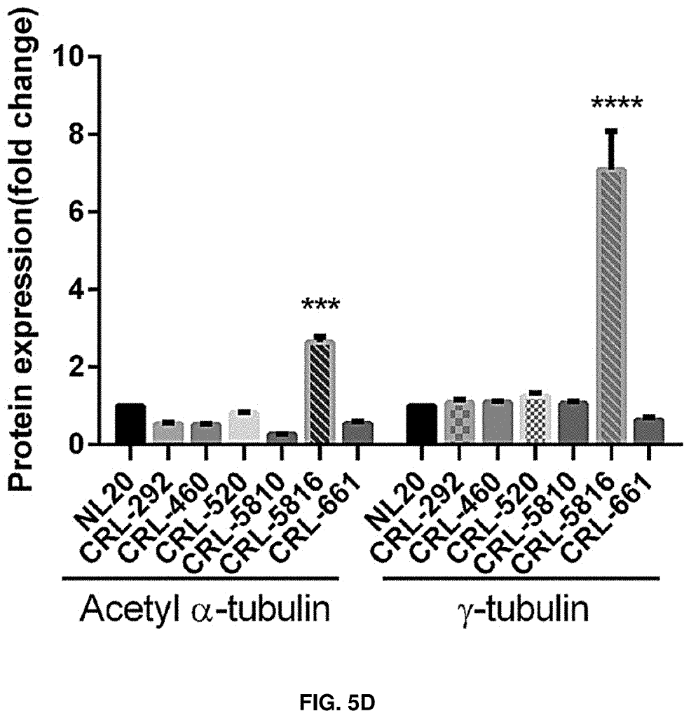

Binding of IQGAP1 to MT bundling proteins and regulation of MT polarity together with current finding that it localizes on amplified centrosomes or undivided (unipolar) centrosomes raised the possibility that centrosome proteins may be used as markers/targets in conjunction with IQGAP1. This was tested by measuring the expression levels in cancer vs. normal cell lines, using immunoblotting (Western Blotting) from total cell lysate. Cell lysate was prepared from cells growing at ˜80% confluency rinsed with ice-cold PBS and scraped into ice-cold NP40 lysis buffer [20 mmol/L Tris (pH 8.0), 137 mmol/L NaCl, 1% NP40, 10% glycerol] supplemented with protease inhibitors (1 mmol/L phenylmethylsulfoxide, 10 μg/mL aprotinin, 10 μg/mL leupeptin), and 3 mmol/L Na₃VO₄. The lysates were cleared by centrifugation, and protein concentration was determined by bicinchoninic acid assay (Pierce, Rockford, Ill.). Equal amounts of proteins were suspended in SDS-PAGE sample buffer, boiled, resolved on gradient SDS-PAGE, and transferred into a PVD nylon membrane. After blocking, the membranes were blotted with primary antibodies for centrin, acetylated α-tubulin, and γ-tubulin, in comparison with actin as a standard in TBST [50 mmol/L Tris (pH 7.4), 150 mmol/L NaCl, 0.05% Tween 20] plus 1% BSA at 4° C. overnight. Following several washes with TBST, the membranes were incubated with horseradish peroxidase-conjugated appropriate secondary antibodies. The specific signals were obtained using the Amersham enhanced chemiluminescent detection system (Arlington Heights, Ill.) or SuperSignal chemiluminescent solution (Thermo-Fisher Scientific), captured with a BioRad GelDoc imager and quantified with Prizm software. These results are shown in FIGS. 5A-5D.

### Example 6—Differential Expression and Activation of Kinases Involved in Stress or Proliferation Signals

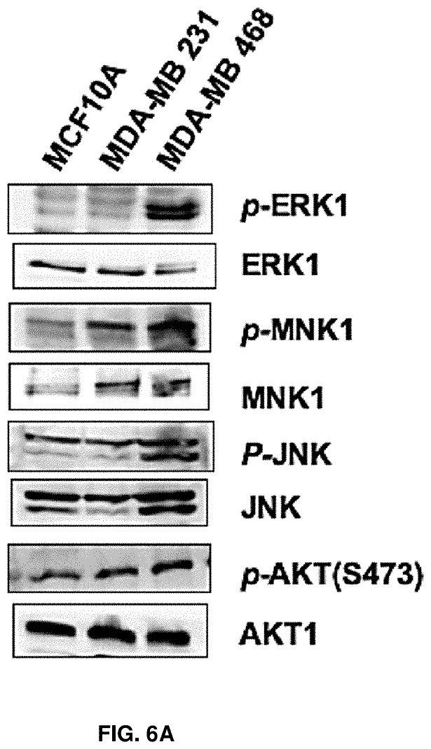

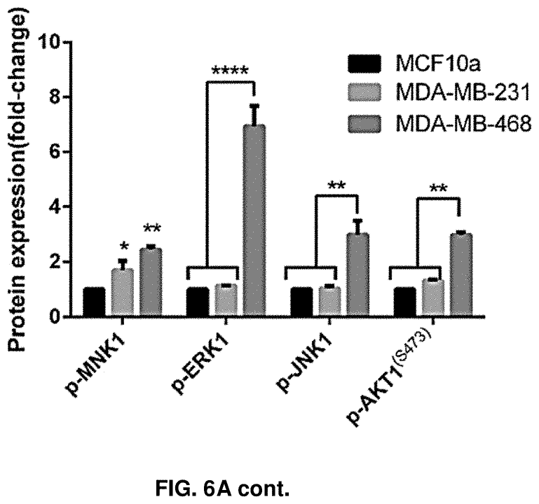

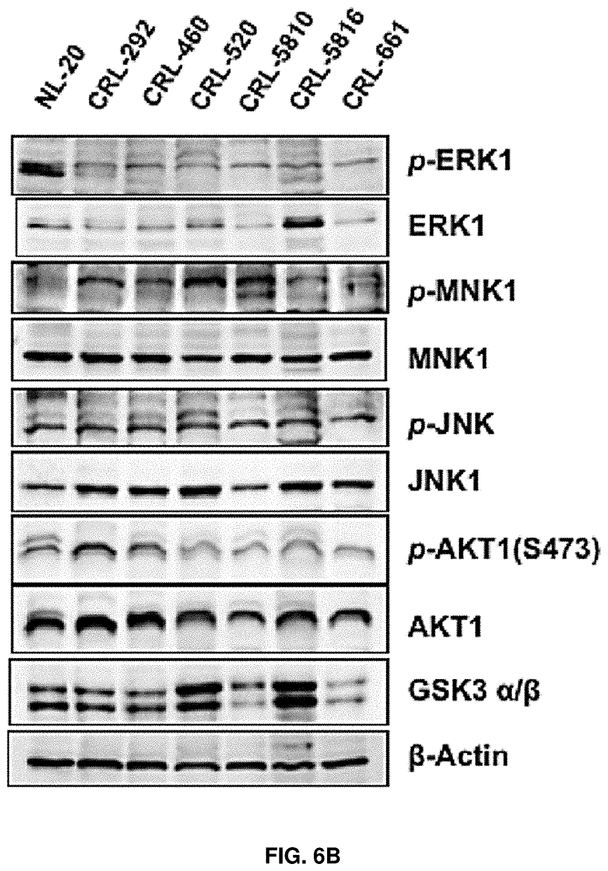

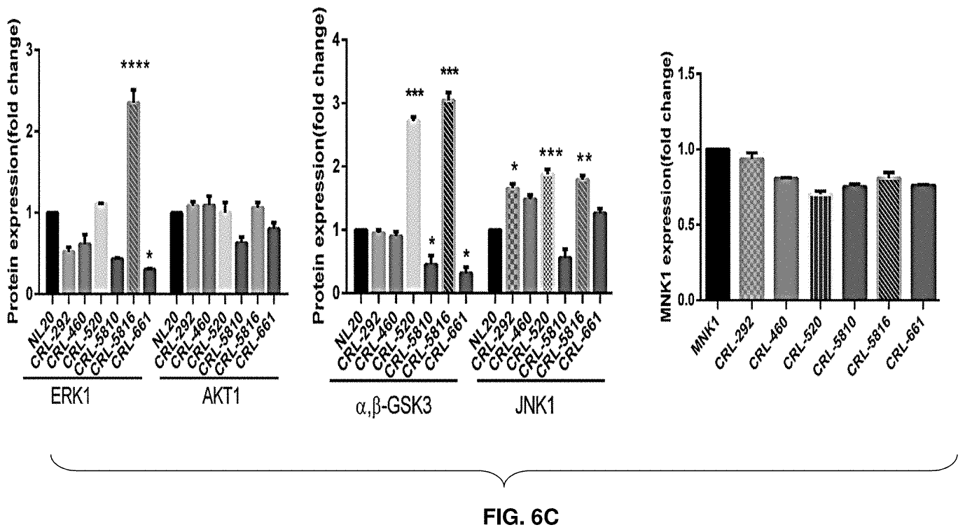

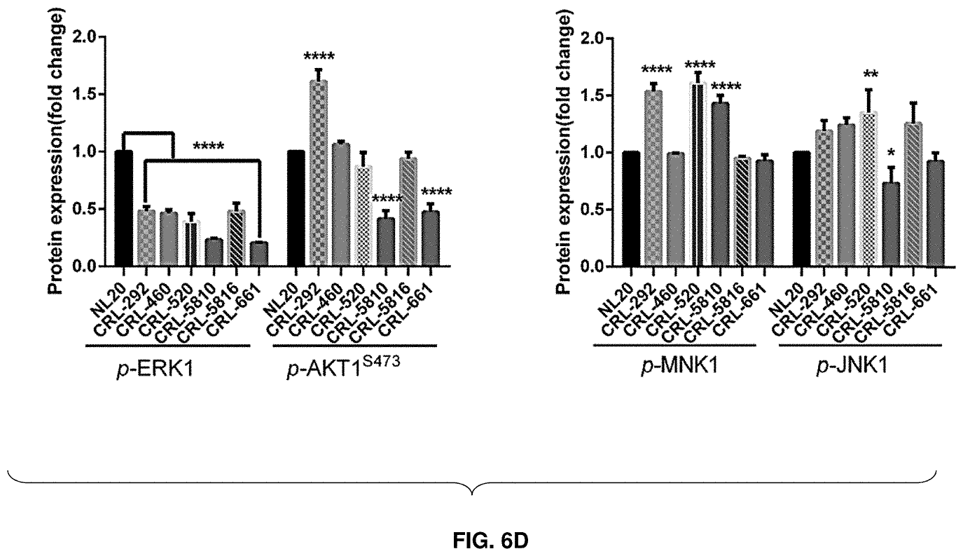

Immunoprecipitation was employed to examine signals downstream of IQGAP1. Because negative mutants of IQGAP1 activate the MAPK Erk1/2, and these mutants behave like the TNBC MDA-MB 468, the phosphorylation mediated activity of MAPK was examined, and the downstream MnK1 was examined by antibodies that detect the phosphorylated form. It is believed that activation of MAPK and the generation of monopolar centrosome may activate stress signals, so the activity of JNK, a hallmark of cellular stress, was determined. Activity of proliferation signal through Akt1 was also determined because IQGAP1 mediates cell proliferation via S6K-Akt1. Immunoblotting was used as detailed above and validated antibodies from Santa Cruz or Cell Signaling Biotech. The results show that these kinases are differentially activated in different lung and breast cancer cell lines, and can be used to differentiate/classify different cell lines isolated from different patients. These markers also are useful in distinguishing cancer cells from two different racial groups. These results are shown in FIGS. 6A-6D.

### Example 7—Differential Expression of the β-Catenin and Nrf1 Transcription Factors Downstream of IQGAP1 Signaling

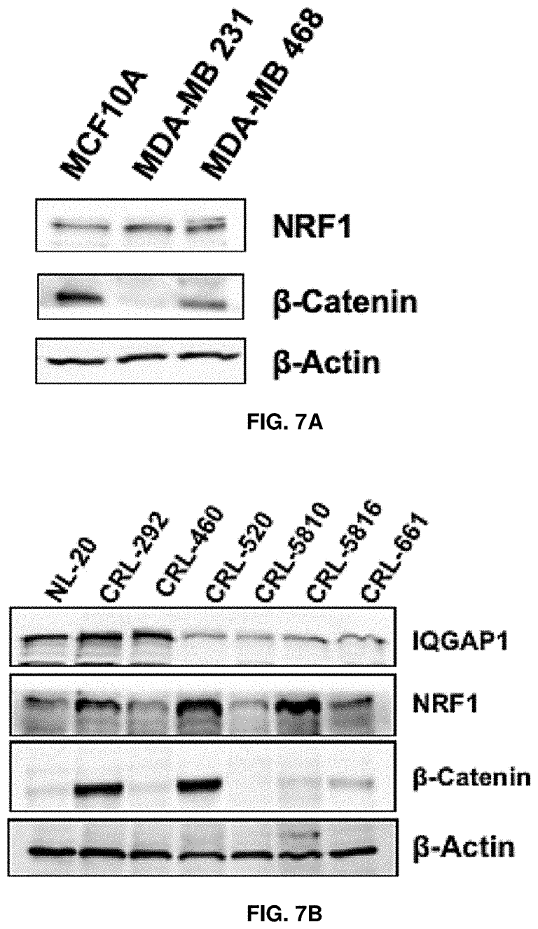

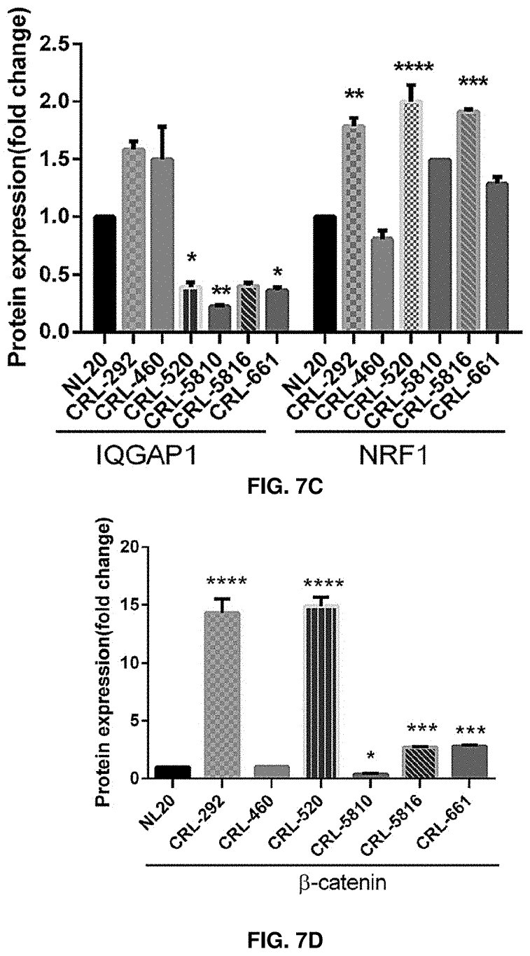

Activation of MAPK, Mnk1, or Akt1 leads to gene expression through activating transcription factors. IQGAP1 binds β-catenin and co-activates transcription in the nucleus. Nrf1 is a transcription factor associated with cancer. Expression of β-catenin and Nrf1 was measured by immunoblotting as described above using antibodies specific for the two proteins. Two transcription factors differentially activated in the different cancer cell lines were found, indicating that they can be used as classification markers and personalized therapeutic targets. These results are shown in FIGS. 7A-7D.

### Example 8—Differential Expression of Upstream the 2-Alpha Adrenergic Receptor (2-AAR)

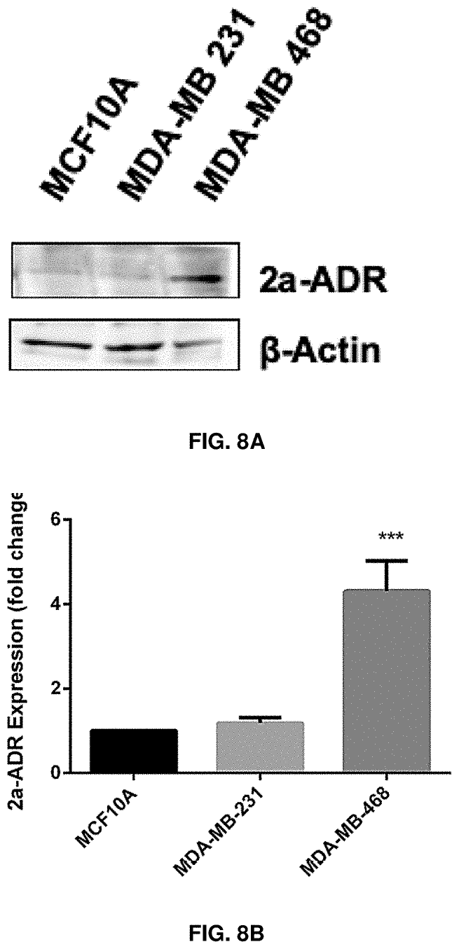

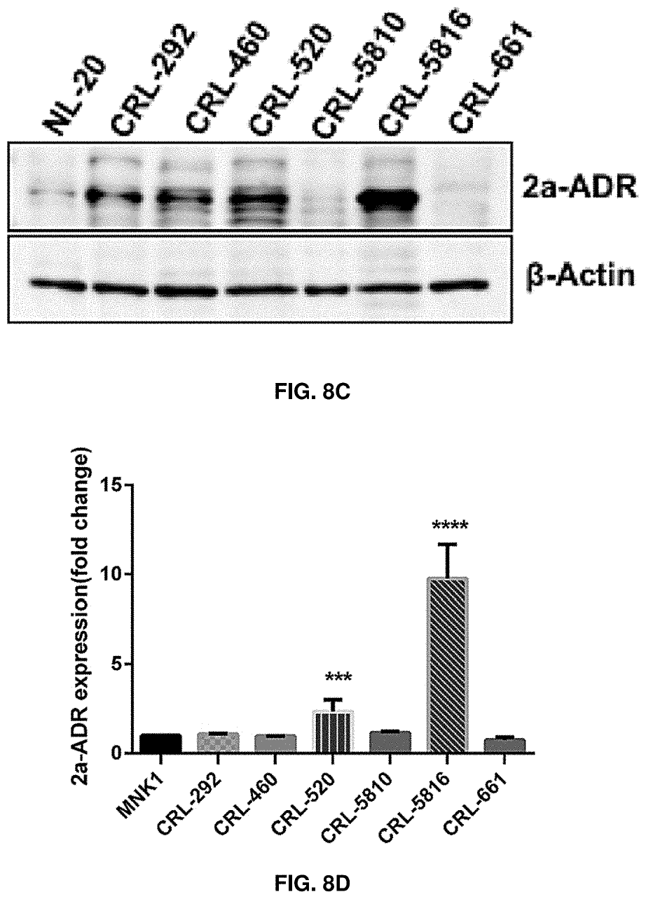

The oncoprotein IQGAP1 widely binds an array of receptor in a ligand specific manner to generate specific downstream effects. The 2-alpha adrenergic (2-AAR) receptors were reported to be expressed on tumor cells and thus may be a clinical target. Using immunoblotting method described above from cell lysate isolated from TNBC and lung cancer obtained from Caucasian and African American females and males, respectively, along with antibodies specific for 2-AAR, it was found that the receptor is differentially expressed in cancer, meaning that while some cancer cell lines highly express 2-AAR, others do not. This finding, shown in FIGS. 8A-8D, provides a method for differentiating between individual cancer regardless of organ site or gender. Ligands for the receptor exert effects on brain disorders such as depression, schizophrenia, Alzheimer's disease, Parkinson's disease, amnesia, and stroke, but the mechanisms of the drugs is unknown. These drugs can be repurposed for treating cancers defined by 2-AAR expression.

### Example 9

### Physical Interaction of IQGAP1 with BRCA1, Nrf1, Mnk1, and 2-ADR

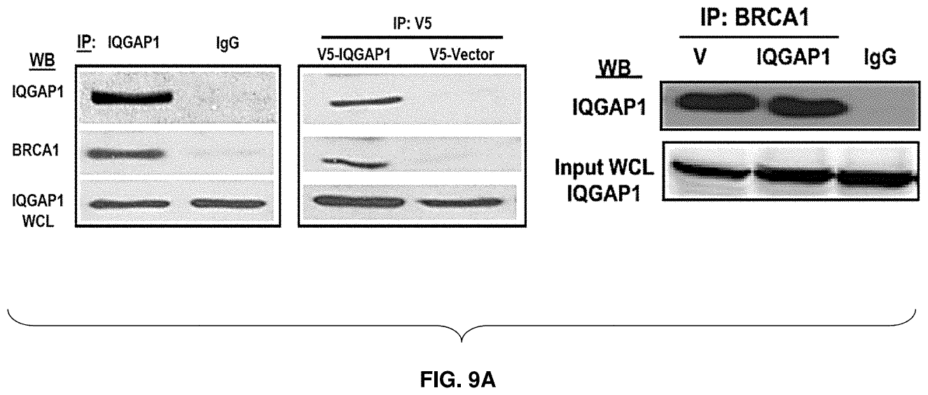

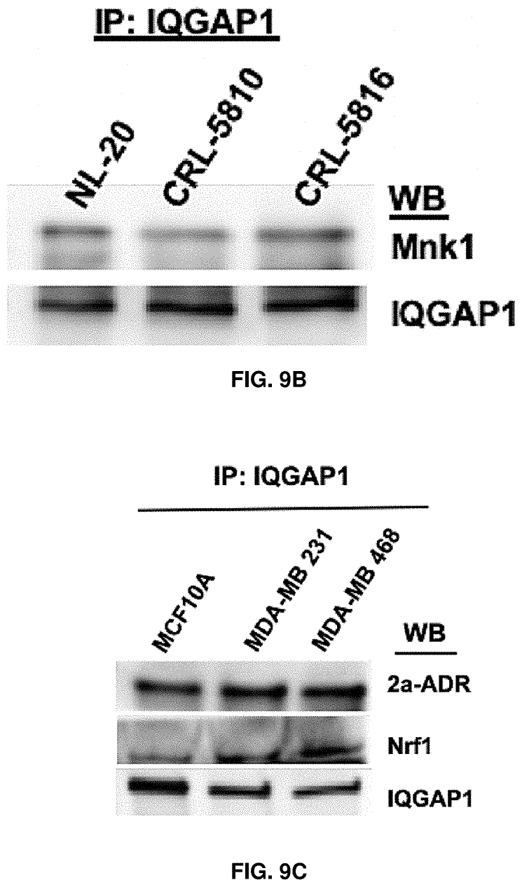

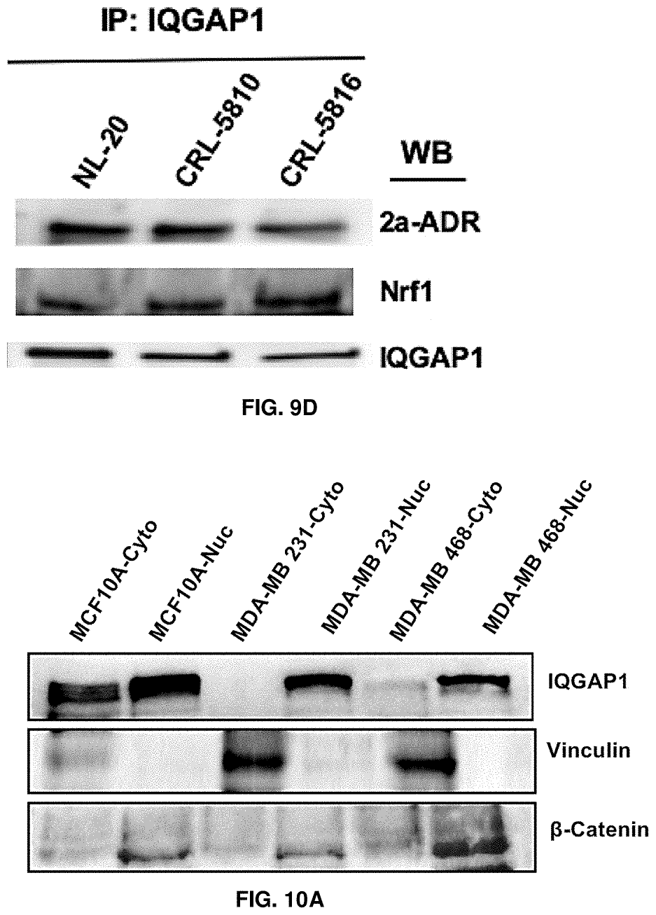

To provide evidence that these markers act with the IQGAP1 pathway, physical association of each marker was evaluated in control and cancer cell lines using immunoprecipitation (IP). Cells growing at ˜80% confluency were lysed as described above. The lysates were cleared by centrifugation, and protein concentration was determined by bicinchoninic acid assay (Pierce, Rockford, Ill.). Four hundred to 1,000 μL of lysates were precleared with 15 μl of PBS-equilibrated protein G or A beads for 1 hr. and used for immunoprecipitation reaction with specific antibodies against the proteins at 4° C. overnight with back-to-back rotation followed by incubation with protein-A/G-Sepharose for 2 hours at 4° C. to collect the immune complexes. The beads were washed with NP40 buffer five times, and analyzed by SDS-PAGE and Western blotting (immunoblotting) as described above. The detected physical interaction indicates that these proteins for a molecular signature are helpful in cancer classification and personalized medicine. These results are shown in FIGS. 9A-9D. Furthermore, drugs against 2-AAR, Mnk1, Nrf1, and BRCA1 are useful in targeting tumors marked by specific expression of these markers when combined with IQGAP1 mislocalization and/or activity.

### Example 10—Inappropriate Stabilization of Sub-Cellular Location of Oncogenic IQGAP1 and β-Catenin Signal

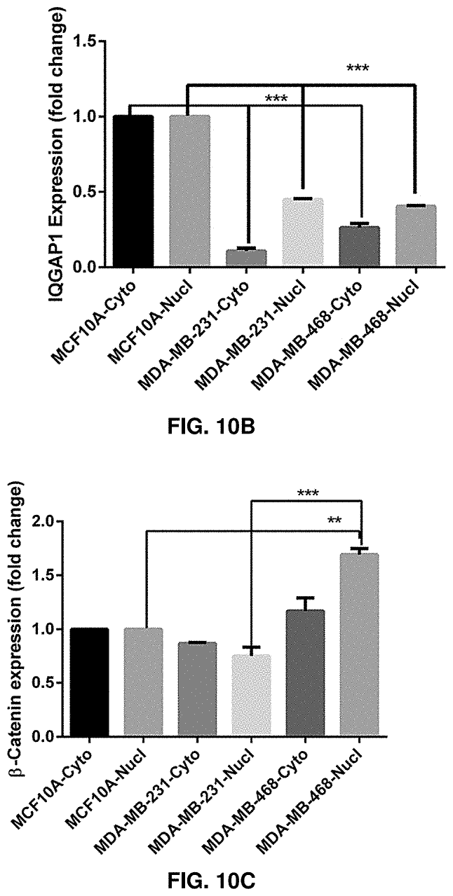

For normal function, IQGAP1 must cycle between phospho- and unphospho-form and shuttles between cytoplasm and nucleus. It has been previously shown that stabilization of any form leads to disease. A biochemical fractionation method, detailed above, was used to measure the levels of IQGAP1 and β-catenin in the nuclei of normal and cancer cell lines against vinculin as a cytoplasmic standard and PARP as a nuclear standard to ensure clean fractions. In normal cells, the two proteins are found both in the cytoplasm and the nucleus. These results are shown in FIGS. 10A-10C. Stabilization of the two proteins in the nuclei of cancer cells indicates a sustained proliferation signal that promotes or sustains cancer cell growth and can be a clinical target as well as a diagnostic marker.

### Example 11—Interaction and Localization of IQGAP1 and BRCA1

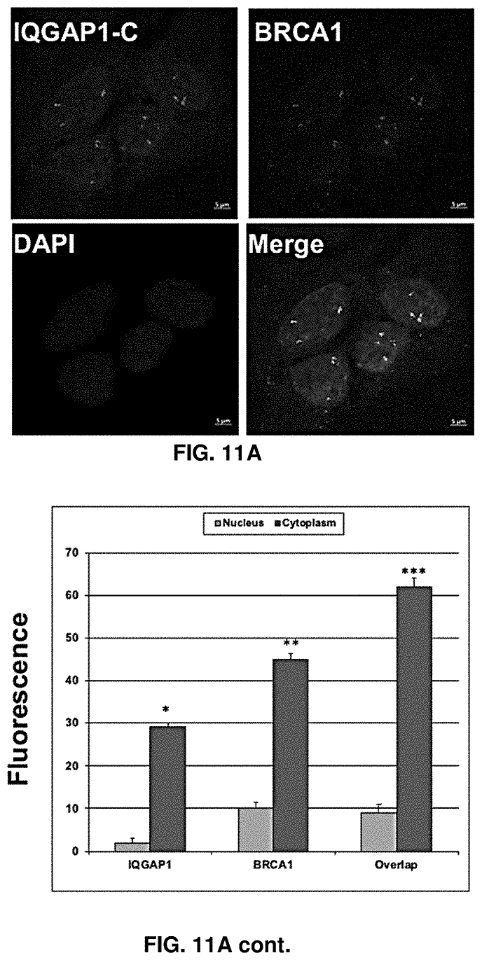

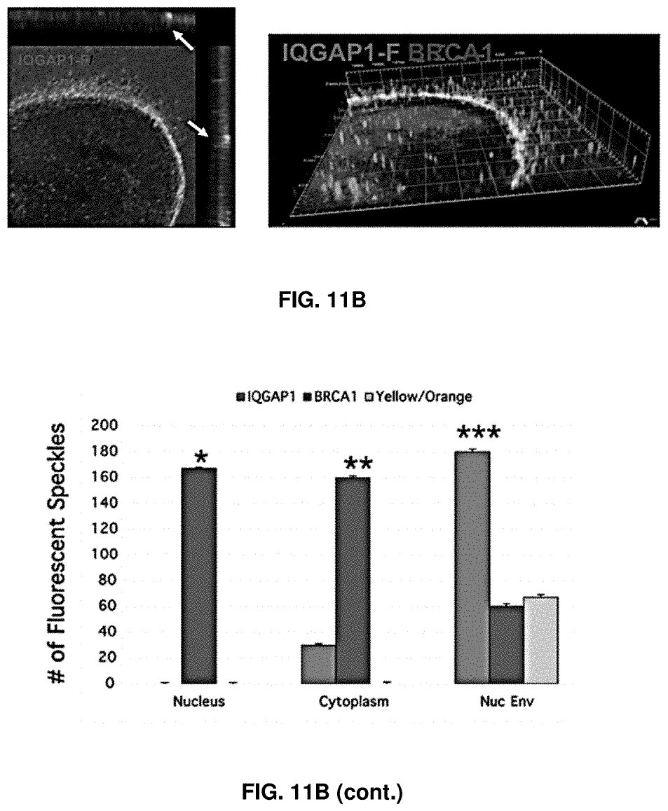

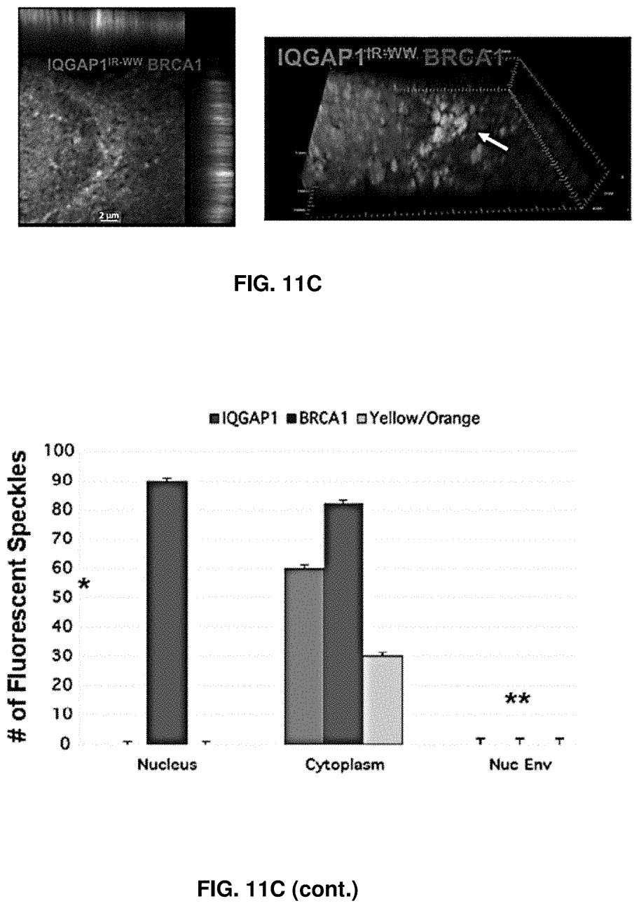

As shown in FIGS. 11A-11C, it was found that IQGAP1 interacts and colocalizes with BRCA1 and affects BRCA1 subcellular distribution.

### Example 12—Localization and Expression of IQGAP1 and BRCA1 in Human Tissues

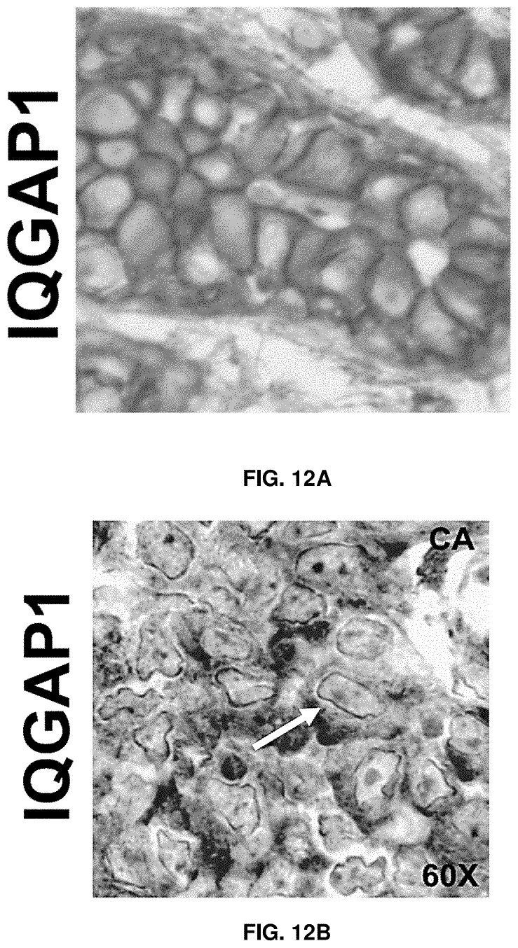

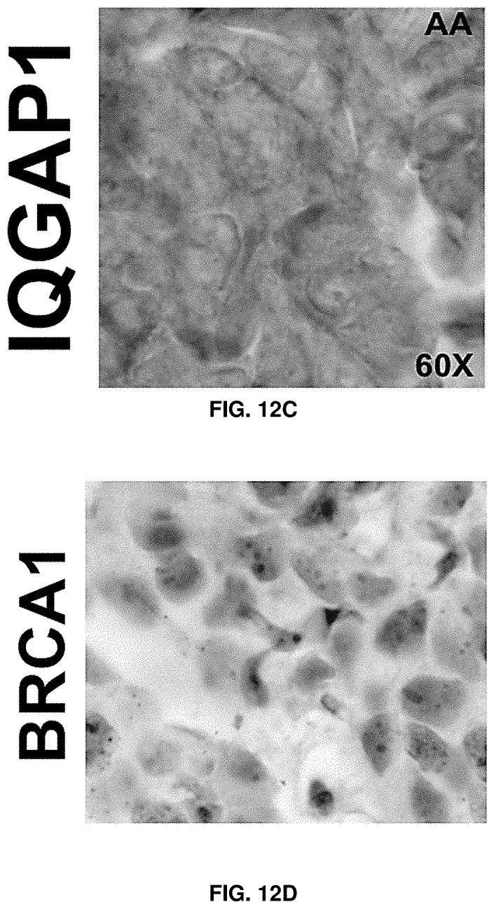

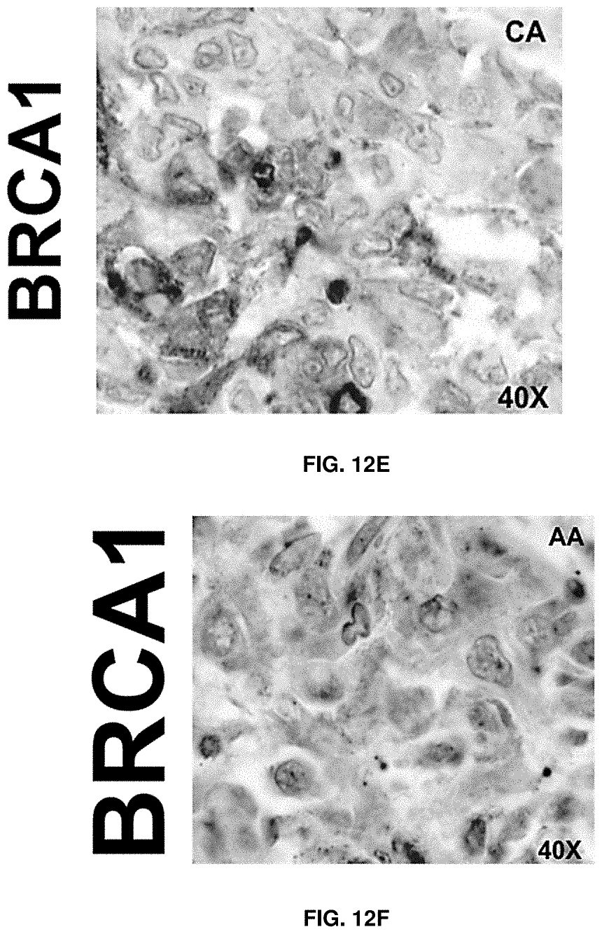

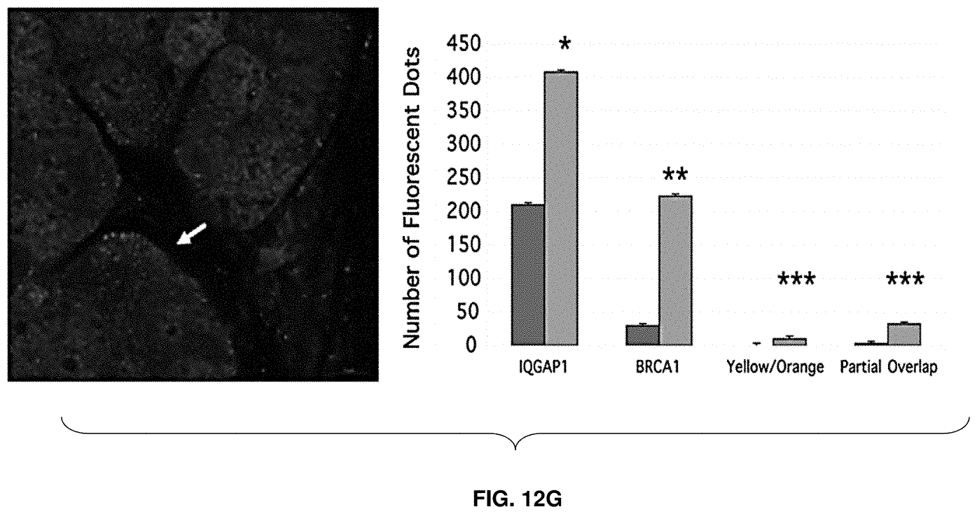

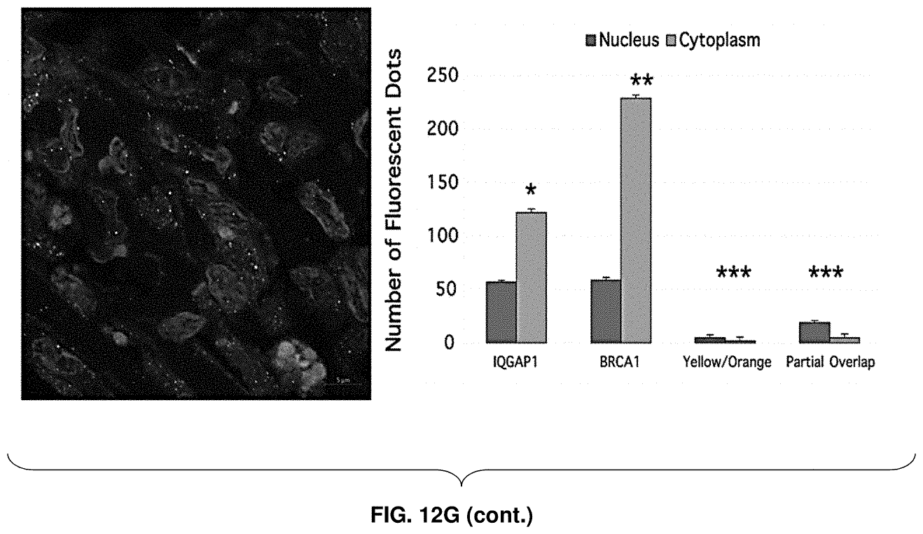

As seen in FIGS. 12A-12G, IQGAP1 is found in the plasma membrane (cell peripheries) in normal tissues, in CA tissues is paranuclear (in nuclear envelope), and in aggregates in the cytoplasm. In AA, IQGAP1 is dispersed in the cytoplasm. BRCA1 is mostly nuclear in normal tissues and found in cytoplasmic aggregates in cancer similar to IQGAP1. These results are similar to those in cell culture where IQGAP1 and BRCA1 co-localize in cytoplasmic aggregates or in the nuclear envelope in corresponding cells. (FIG. 12G.)

### Example 13—Expression Levels of Markers

Expression levels of several markers were analyzed in the cancer cell lines MDA-MB-231, MDA-MB-468, CRL-292, CRL-460, CRL-520, CRL-5810, CRL-5816, and CRL-661. The results are shown in Table 1 (FIG. 13). As seen in FIG. 13, 18 markers show dysregulated expression levels in the cancer cell lines.

### Example 14—IQGAP1 Modulates Apoptosis (Programmed Cell Death) to Activate Bcl2 During Tumorigenesis

The mechanism of IQGAP1 in tumorigenesis was substantiated in preclinical mouse model. In cell culture active IQGAP1 behaves similar to MDA-MB-231 in terms of signaling and centrosome phenotype and, thus, were compared in mice.

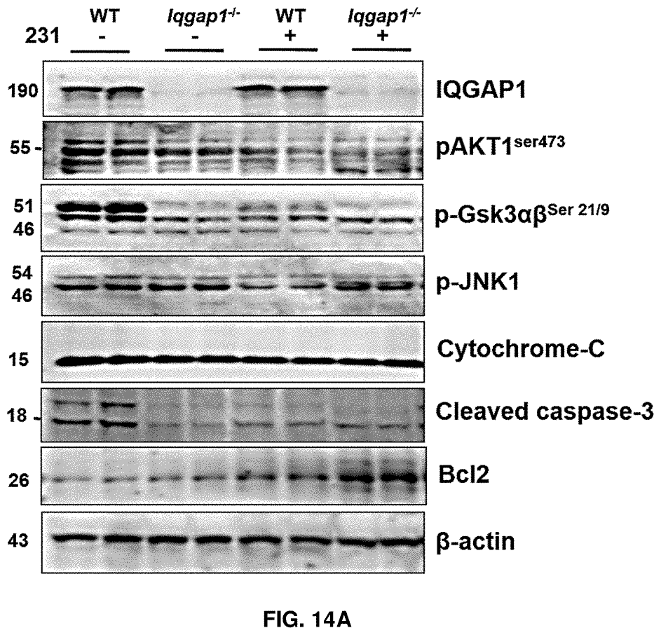

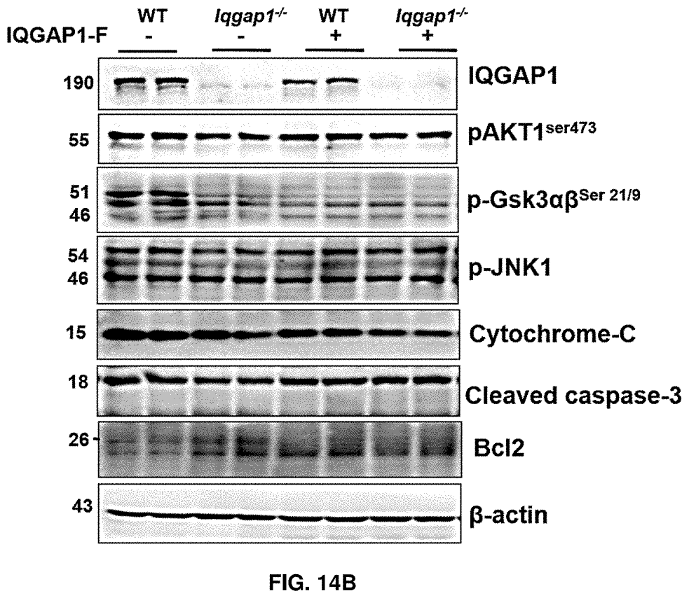

A single dose (one million cells) of IQGAP1-F and MDA-MB-231 cells were separately injected in mammary gland fat pad of wild type (WT) and iqgap1^{-/-} knockout (KO) mice. Six-week later, total extracts from different organs including mammary tissues was compared for IQGAP1 signaling and apoptotic pathways by immunoblot (Western blotting), as shown in FIGS. 14A-14B.

Compared to WT, IQGAP1 KG elevates Bcl2 expression, reduces cytochrome C level and caspase 3 activity (FIGS. 14A-14B left two bars).

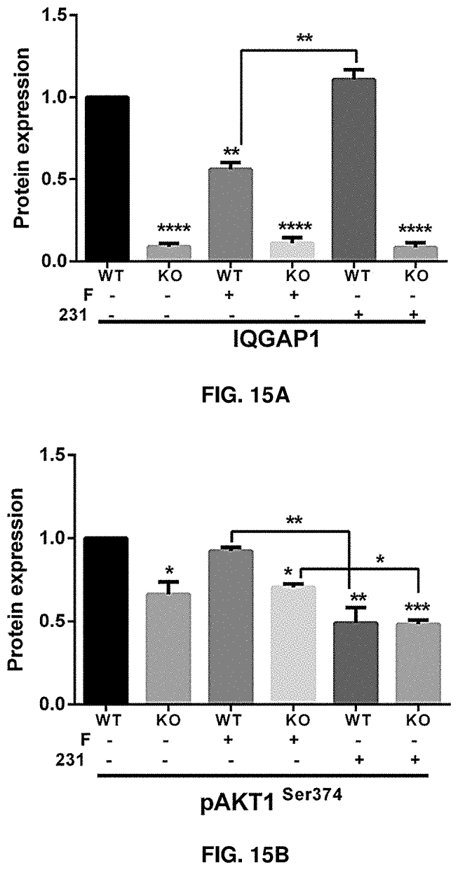

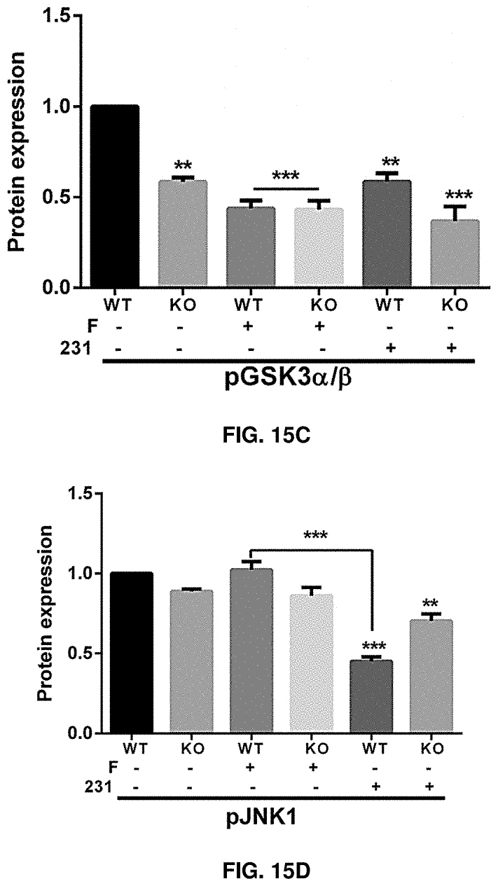

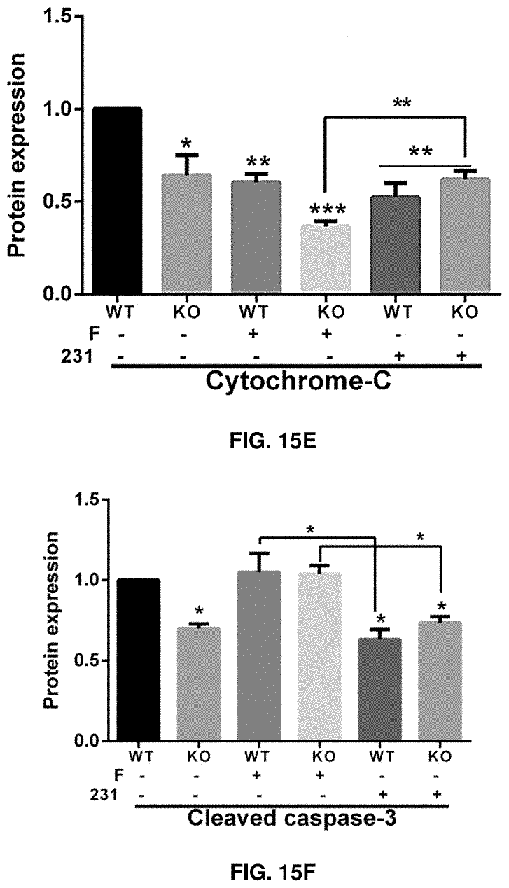

FIGS. 15A-15E show protein expression for IQGAP1, pAKT1, pGSK3α/β, pJNK1, Cytochrome-C, and cleaved caspase-3, respectively.

Overall, injection of either IQGAP1-F or 231 triple negative breast cancer (TNBC) cells induced similar signaling events, including JNK activation in KO (FIG. 15D).

Injection of IQGAP1-F further reduced cytochrome C level but increased caspase 3 activity in KO (FIGS. 15E-15F), showing that IQGAP1 modulates caspase 3 activity. Activated caspase 3, which is an executioner death effector, can produce mitogenic signal in undead cells and induce proliferation, thus explaining the increase in Bcl2 level. B-cell lymphoma 2 (Bcl2) is an oncogene that normally acts as anti-apoptotic by controlling mitochondrial permeability and release of cytochrome C.

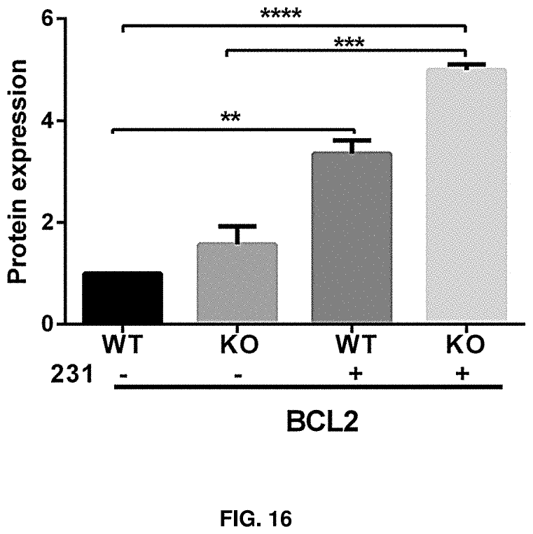

Suppression of Bcl2 leads to release of cytochrome C into the cytosol which accelerates apoptosis by caspases activation. FIG. 16 shows that over-expression of Bcl2 promotes cell survival by suppressing apoptosis, which can lead to protein aggregates associated cancer and observed in variants of TNBC cells and patient tissues.

These data clearly indicate that IQGAP1 modulates apoptosis by which it underlies tumorigenesis and that increase in the activities of IQGAP1 plus that of Bcl2 and caspase 3 together can serve as neoplastic markers in certain cancers like variants of TNBC.

### Example 15—Methods of Treatment

In another aspect, described herein is a method of treating a patient. Such method generally includes: extracting a tissue sample from a patient; analyzing the tissue sample for a change in IQGAP1 expression compared to a control of normal tissue, wherein said change in IQGAP1 expression in the tissue sample compared to the control is indicative of the patient having, or being likely to have, a cancer; and, treating the patient with a drug that modulates expression of IQGAP1.

In certain embodiments, the change in IQGAP1 expression compared to the control comprises one or more of:

i) determining phosphorylation of IQGAP1 to determine whether IQGAP1 is aberrantly phosphorylated in the tissue sample compared to a control of normal tissue; wherein aberrant phosphorylation of IQGAP1 in the tissue sample compared to the control is indicative of the patient having, or being likely to have, a cancer; and,

ii) localization of IQGAP1 to determine whether IQGAP1 is localized in centrosomes or is mislocalized in the tissue sample; wherein mislocalization of IQGAP1 in the tissue sample is indicative of the patient having, or being likely to have, a cancer.

In certain embodiments, the method further comprises: analyzing the tissue sample for an expression level of at least one marker selected from the group consisting of: Bcl2, caspase-3, centrin-2, acetyl α-tubulin, γ-tubulin, NRF1, β-catenin, pERK1, ERK1/2, pMMNk1, MNK1, pJNK, JNK1, pAkt1, Akt1, IQGAP1, pIQGAP1, BRCA1, and 2a-ADR.

In certain embodiments, the method further analyzing the tissue sample for expression levels of at least two of: Bcl2, caspase-3, and IQGAP1.

In certain embodiments, the cancer is triple negative breast cancer in a patient, and the method further comprises:

a) detecting the expression pattern of a set of markers in a sample from the patient, wherein the set of markers comprises two or more markers selected from the group consisting of: Bcl2, caspase-3, centrin-2, acetyl α-tubulin, γ-tubulin, NRF1, β-catenin, pERK1, ERK1/2, pMMNk1, MNK1, pJNK, JNK1, pAkt1, Akt1, IQGAP1, pIQGAP1, BRCA1, and 2a-ADR;

b) diagnosing the patient as needing a cancer therapy regimen when two or more of said markers are expressed; and,

c) treating the patient with an immunotherapeutic that targets the set of markers expressed in the patient.

In certain embodiments, the treating comprises administering to the patient an effective amount of IQGAP1-IR-WW peptide or a drug with same effect.

In certain embodiments, the drug comprises at least one of a kinase inhibitor; an antidepressant, paclitaxel, vinorelbine, docetaxel, and vinblastine.

### Example 16—Identifying Eligibility of Treatments

In another aspect, described herein is a method of identifying a patient as eligible for IQGAP1-directed therapy and administering the IQGAP1 directed therapy to a patient thereby identified as eligible.

When the patient has triple negative breast cancer, the method generally includes:

fixing a tissue sample obtained from the patient, wherein the sample comprises cells of the cancer or carcinoma;

contacting the fixed tissue sample with an anti-IQGAP1 therapeutic agent; and,

detecting binding of the agent to the fixed tissue sample to determine IQGAP1 is expressed in the sample.

The patient is identified as eligible for IQGAP1-directed therapy based on the expression of IQGAP1 as compared to a control.

Thereafter, the patient can be treated by administering the IQGAP1-directed therapy to the patient identified as eligible, wherein the IQGAP1 directed therapy is therapy with an anti-IQGAP1 therapeutic agent.

In certain embodiments, the tissue sample expresses IQGAP1, and IQGAP1 expression is determined as a percentage of tumor cells in the sample expressing detectable IQGAP1.

In certain embodiments, the method further comprises determining a treatment protocol for the patient.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A method of analyzing a sample, the method comprising:
extracting a tissue sample from a patient; and
analyzing the tissue sample for expression levels of two or more markers selected from the group consisting of: Bcl2, caspase-3, centrin-2, acetyl α-tubulin, γ-tubulin, NRF1, β-catenin, pERK1, ERK1/2, pMMNk1, MNK1, pJNK, JNK1, pAkt1, Akt1, IQGAP1, pIQGAP1, BRCA1, and 2a-ADR.

2. The method of claim 1, comprising analyzing the tissue sample for expression levels and/or activation of at least two of: Bcl2, caspase-3, and IQGAP1.

3. The method of claim 1, comprising analyzing the tissue sample for expression levels of each of: centrin-2, pERK1, ERK1/2, MNK1, pAkt1, Akt1, IQGAP1, and BRCA1.

4. The method of claim 1, comprising analyzing the tissue sample for expression levels of each of: acetyl α-tubulin, γ-tubulin, NRF1, β-catenin, pMMNk1, pJNK, JNK1, pIQGAP1, and 2a-ADR.

5. The method of claim 1, comprising analyzing the tissue sample for expression levels of three, four, five, six, seven, eight, nine, or ten more of the markers.

6. The method of claim 1, wherein analysis of IQGAP1 includes determining one or more of whether IQGAP1 is mislocalized in the tissue sample, or is aberrantly phosphorylated in the tissue sample.

7. A method of analyzing a sample, the method comprising:
extracting a tissue sample from a patient;
analyzing the tissue sample for a change in IQGAP1 expression compared to a control;
and,
analyzing the tissue sample for an expression level of at least one marker selected from the group consisting of: Bcl2, caspase-3, centrin-2, acetyl α-tubulin, γ-tubulin, NRF1, β-catenin, pERK1, ERK1/2, pMMNk1, MNK1, pJNK, JNK1, pAkt1, Akt1, IQGAP1, pIQGAP1, BRCA1, and 2a-ADR.

8. The method of claim 7, wherein the change in IQGAP1 expression comprises one or more of:
aberrant phosphorylation of IQGAP1;
IQGAP1 localized in centrosomes; and,
IQGAP1 mis-localized in the tissue sample.

9. The method of claim 7, wherein the sample is analyzed for the present of triple negative breast cancer (TNBC), and the method further comprises:
distinguishing between distinct variants of TNBC,
wherein said distinct variants include Caucasian (CA) TNBC, and African American (AA) TNBC.

10. A method of treating a patient, the method comprising:
extracting a tissue sample from a patient;
analyzing the tissue sample for a change in IQGAP1 expression compared to a control of normal tissue,
wherein said change in IQGAP1 expression in the tissue sample compared to the control is indicative of the patient having, or being likely to have, a cancer; and
treating the patient with a drug that modulates expression of IQGAP1.

11. The method of claim 10, wherein the change in IQGAP1 expression compared to the control comprises one or more of:
i) determining phosphorylation of IQGAP1 to determine whether IQGAP1 is aberrantly phosphorylated in the tissue sample compared to a control of normal tissue;
wherein aberrant phosphorylation of IQGAP1 in the tissue sample compared to the control is indicative of the patient having, or being likely to have, a cancer; and,
ii) localization of IQGAP1 to determine whether IQGAP1 is localized in centrosomes or is mislocalized in the tissue sample;
wherein mislocalization of IQGAP1 in the tissue sample is indicative of the patient having, or being likely to have, a cancer.

12. The method of claim 10, further comprising:
analyzing the tissue sample for an expression level of at least one marker selected from the group consisting of: Bcl2, cleaved caspase-3, centrin-2, acetyl α-tubulin, γ-tubulin, NRF1, β-catenin, pERK1, ERK1/2, pMMNk1, MNK1, pJNK, JNK1, pAkt1, Akt1, IQGAP1, pIQGAP1, BRCA1, and 2a-ADR.

13. The method of claim 12, comprising analyzing the tissue sample for expression levels of at least two of: Bcl2, cleaved caspase-3, and IQGAP1.

14. The method of claim 10, wherein the cancer is triple negative breast cancer in a patient, and the method comprising:
a) detecting the expression pattern of a set of markers in a sample from the patient, wherein the set of markers comprises two or more markers selected from the group consisting of: Bcl2, caspase-3, centrin-2, acetyl α-tubulin, γ-tubulin, NRF1, β-catenin, pERK1, ERK1/2, pMMNk1, MNK1, pJNK, JNK1, pAkt1, Akt1, IQGAP1, pIQGAP1, BRCA1, and 2a-ADR;
b) diagnosing the patient as needing a cancer therapy regimen when two or more of said markers are expressed; and
c) treating the patient with an immunotherapeutic that targets the set of markers expressed in the patient.

15. The method of claim 10, wherein the treating comprises administering to the patient an effective amount of IQGAP1-IR-WW peptide or a drug with same effect.

16. The method of claim 10, wherein the drug comprises at least one of a kinase inhibitor; an antidepressant, paclitaxel, vinorelbine, docetaxel, and vinblastine.

17. A method of identifying a patient as eligible for IQGAP1-directed therapy and administering the IQGAP1 directed therapy to a patient thereby identified as eligible, the method comprising:
fixing a tissue sample obtained from the patient, wherein the sample comprises cells of the cancer or carcinoma,
contacting the fixed tissue sample with an anti-IQGAP1 therapeutic agent, and
detecting binding of the agent to the fixed tissue sample to determine IQGAP1 is expressed in the sample, and
identifying the patient as eligible for IQGAP1-directed therapy based on the expression of IQGAP1 as compared to a control; and
administering the IQGAP1-directed therapy to the patient identified as eligible, wherein the IQGAP1 directed therapy is therapy with an anti-IQGAP1 therapeutic agent.

18. The method of claim 17, wherein the tissue sample expresses IQGAP1, and IQGAP1 expression is determined as a percentage of tumor cells in the sample expressing detectable IQGAP1.

19. The method of claim 17, further comprising determining a treatment protocol for the patient, wherein detectable expression of IQGAP1 is an indication that the treatment protocol include treatment with the IQGAP1-directed therapy.

20. The method of claim 17, wherein the patient has triple negative breast cancer (TNBC).
